SEQUENCE LISTING (9 sequences)

SEQ ID NO: 1 - DNA, 19 bp, Artificial Sequence
  GFP ds siRNA Sense strand (ss)
gcagcacgacuucuucaag

SEQ ID NO: 2 - DNA, 20 bp, Artificial Sequence
  GFP ds siRNA Antisense strand (as)
cuugaagaagucgugcugct

SEQ ID NO: 3 - DNA, 21 bp, Artificial Sequence
  GFP mRNA Target Site Sequence
aagcagcacgacuucuucaag

SEQ ID NO: 4 - DNA, 21 bp, Artificial Sequence
  RFP mRNA Target Site Sequence
aagugggagcgcgugaugaac

SEQ ID NO: 5 - DNA, 19 bp, Artificial Sequence
  RFP ds siRNA Sense strand (ss)
gugggagcgcgugaugaac

SEQ ID NO: 6 - DNA, 20 bp, Artificial Sequence
  RFP ds siRNA Antisense strand (as)
guucaucacgcgcucccact

SEQ ID NO: 7 - DNA, 21 bp, Artificial Sequence
  CDK9 mRNA Target Site Sequence
aaccaaagcuucccccuauaa

SEQ ID NO: 8 - DNA, 19 bp, Artificial Sequence
  CDK9 ds sIRNA Sense strand (ss)
ccaaagcuucccccuauaa

SEQ ID NO: 9 - DNA, 20 bp, Artificial Sequence
  CDK9 ds sIRNA Antisense strand (as)
tgguuucgaagggggauauu

(19) United States
(12) Patent Application Publication
(10) Pub. No.: US 2004/0204420 A1
(43) Pub. Date: Oct. 14, 2004

(54) Compounds for modulating RNA interference

(76) Inventor: Tariq M. Rana, Shrewsbury, MA (US)

Correspondence Address: LAHIVE & COCKFIELD, LLP., 28 STATE STREET, BOSTON, MA, 02109, US

(21) Appl. No.: 10/635,849

(22) Filed: Aug. 5, 2003

Related U.S. Application Data
(60) Provisional application No. 60/401,174, filed on Aug. 5, 2002.
(60) Provisional application No. 60/489,590, filed on Jul. 22, 2003.
(60) Provisional application No. 60/489,960, filed on Jul. 21, 2003.

(51) Int. Cl. A61K 31/525; C07D 475/02

(52) U.S. Cl. 514/251; 544/258

(57) ABSTRACT

The present invention pertains to compounds effective at modulating (inhibiting or enhancing) RNA interference in a cell or organism. Featured compounds are set forth and exemplified herein. Therapeutic methods and pharmaceutical compositions featuring the compounds are also provided. The invention further pertains to knock-out or knock-down cells and organisms including the compounds, and methods of analysis of gene expression profiles and proteomes.

### RELATED APPLICATIONS

This application claims the benefit of U.S. Provisional Patent Application Serial Nos. 60/401,174, filed Aug. 5, 2002, entitled “Small Molecule Inhibitors of RNAi,” 60/XXX,XXX, filed Jul. 22, 2003 and 60/489,960, filed, Jul. 23, 2003, both entitled “Compounds for Modulating RNA Interference,” the entire contents of which applications are incorporated herein by this reference.

### GOVERNMENT RIGHTS

This invention was made at least in part with support under grant numbers AI41404 and AI43198, awarded by the United States National Institutes of Health and the National Institute of Allergy and Infectious Diseases.

### BACKGROUND OF THE INVENTION

Double stranded RNA (dsRNA) induces a sequence-specific degradation of homologous mRNA in the cellular process known as RNA interference (RNAi). DsRNA-induced gene silencing has been observed in evolutionarily diverse organisms such as nematodes, flies, plants, fungi, and mammalian cells. Although the entire mechanism of RNAi has not yet been elucidated, several key elements have been identified. RNAi is initiated by an ATP-dependent processive cleavage of dsRNA into 21-23 nucleotide short interfering RNAs (siRNAs) by the DICER endonuclease. The siRNAs are then incorporated into an RNA-induced silencing complex (RISC). This protein and RNA complex is activated by ATP-dependent unwinding of the siRNA duplex. The activated RISC utilizes the antisense strand, also referred to as the guide strand, of the siRNA to recognize and cleave the corresponding mRNA, resulting in decreased expression of the protein encoded by the mRNA.

There recently has been a great deal of interest in the use of RNAi for basic research purposes and for the development of therapeutics to treat, e.g., disorders and/or diseases associated with unwanted or aberrant gene expression, however, siRNA effectiveness at mediating RNAi varies greatly, and can be affected by a number of factors including, but not limited to, the size of the siRNA, the size and nature of any overhangs, and the specificity of the siRNA. Even siRNAs having optimal length, overhangs and specificity, can be ineffective at mediating RNAi. Given the potential of therapies and diagnostics utilizing RNAi interference (e.g., for the treatment of HIV and cancer), the utility of RNAi interference in the study of functional genomics, in screening assays, and providing knock-out and/or knock down cells or organisms, there exists a need to enhance mediation of RNAi.

### SUMMARY OF THE INVENTION

The present invention pertains to novel compounds effective at modulating RNA interference (RNAi) and exhibit low cell toxicity. Such compounds have wide-ranging therapeutic potential in treatment of, e.g., diseases and disorders associated with unwanted or aberrant gene expression. The present invention features compounds, compositions and methods for modulating RNAi in vivo and in vitro.

Featured compounds are those corresponding to the formulae set forth herein, and include the compounds depicted in the examples and figures. Therapeutic methods, pharmaceutical compositions, knock-out and/or knock-down cells and organisms, methods of analyzing functional genomics, and methods of analyzing the RNAi modulation pathway and related biological mechanisms, also are provided.

The invention provides a compound of Formula I:

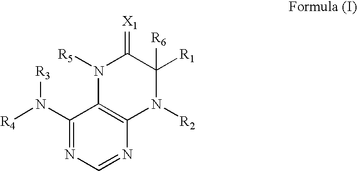

wherein

R¹ is alkyl, alkenyl, or alkynyl, optionally interrupted by one or more O, N, NR^{a}, or S groups, and optionally substituted with one or more hydroxyl, halo, alkoxy, oxo, amino, cycloalkyl, heterocycloalkyl, aryl, heteroaryl, thione or thiol moiety, the cycloalkyl, heterocycloalkyl, aryl, or heteroaryl is optionally substituted with one or more oxo, hydroxyl, thione, thiol or fused ring moiety;

R² is H or C₁₋₆ alkyl, or R¹ and R² together form a 3- to 8-membered ring optionally interrupted by one or more O, NR^{a}, or S and optionally substituted with one or more hydroxyl, halo, alkoxy, oxo, amino, thione or thiol moiety;

R₃ is alkyl, alkenyl, alkynyl, aryl, cycloalkyl, or heterocycloalkyl, optionally interrupted by one or more O, NR^{a}, or S group, and optionally substituted with one or more hydroxyl, alkoxy, halo, aryl, heteroaryl, cycloalkyl, heterocycloalkyl, carboxyl, alkylcarboxy, oxo, amino, aminoaryl, aminoheteroaryl, indole, alkoxyaryl, alkoxycarbonyl, thione, thiol, or a fused ring moiety; and

R₄ is H or C₁₋₆ alkyl optionally substituted with a hydroxyl, halo, or amino group, or R³ and R⁴ together form a 3- to 8-membered ring optionally interrupted by one or more O, NR^{a}, or S and optionally substituted with one or more alkyl, hydroxyl, alkoxy, halo, aryl, heteroaryl, cycloalkyl, heterocycloalkyl, carboxyl, alkylcarboxy, oxo, amino, indole, thione, thiol or a fused ring moiety;

R⁵ is H or C₁₋₆ alkyl;

R⁶ is H or C₁₋₆ alkyl;

X¹ is O, S or NR^{a}; and

R^{a} is H, C₁₋₆ alkyl, or C₁₋₆ acyl,

or a salt thereof.

Other features and advantages of the invention will be apparent from the following detailed description and claims.

### BRIEF DESCRIPTION OF THE DRAWINGS

FIG. 1A-D is a chart illustrating exemplary compounds of the present invention indicating modulation activity.

FIG. 2A is a schematic drawing of dsRNAs (siRNAs) used for targeting GFP mRNA and RFP mRNA. siRNA duplexes were synthesized for targeting sequence positions 238-258 of EGFP mRNA relative to the start codon, and positions 277-297 of RFP mRNA.

FIG. 2B is a set of fluorescence images showing specific RNAi effects on living HeLa cells. Panels a and b are images of mock-treated cells (no siRNA added); panels c and d are images of GFP siRNA-treated cells; and panels e and f are images of RFP siRNA-treated cells.

FIG. 2C is a pair of graphs illustrating the results of quantitative analysis of the RNAi effects observed in FIG. 2B.

FIG. 2D is a pair of graphs depicting the excitation and emission spectra for a number of exemplary fluorescent proteins that can be used in dual fluorescence reporter gene assays, including GFP and RFP.

FIG. 3A is a schematic drawing of dsRNAs (siRNAs) used for targeting CDK9 mRNA. The position of the first nucleotide of the mRNA target site is indicated relative to the start codon of CDK9 mRNA. The sequence of the antisense strand of siRNA is complementary to the mRNA target site.

FIG. 3B is a Western blot of proteins from an experiment testing the ability of ATPA18, ATPA18-1, and ATPA18-2 to inhibit CDK9 protein expression. The membrane was first probed with antibodies raised against CDK9. CycT1 was also detected as a control showing that RNAi effect was specific for CDK9 mRNA degradation.

FIG. 3C is a Western blot of proteins from an experiment testing the ability of ATPA21 to inhibit CDK9 protein expression. The membrane was first probed with antibodies raised against CDK9. CycT1 was also detected as a control showing that RNAi effect was specific for CDK9 mRNA degradation.

FIG. 4A depicts a model for RNAi in human cells highlighting the role of ATP.

FIG. 4B is a graph depicting the results of a screening of candidate compounds as modulators of RNAi in human cells. Structures of the two compounds exhibiting inhibition effects on RNAi also are depicted.

FIG. 4C includes two immunoblots demonstrating the effect of ATPA-18 and ATPA-21 on RNAi targeted to endogenously expressed gene product, CDK9.

FIG. 5A is a chart demonstrating the effect of ATPA-18 and ATPA-21 on the RNAi pathway at 0 h and 6 h post-transfection as quantified by the dual fluorescence assay.

FIG. 5B depicts localization patterns of siRNAs with and without addition of an RNAi inhibitor (ATPA18).

FIG. 5C depicts the results of a biotin pull-out assay indicating that helicase activity, and not kinase activity, was the cellular target for small molecular inhibitors of RNAi in human cells.

FIG. 6A depicts a model for measuring acceptor emission dectection using a FRET filter.

FIG. 6B is a series of images of the fluorescence emission signal and FRET signal of labeled siRNA transfected into HeLa cells with and without ATPA-18 at 0 and 12 h after transfection.

FIG. 7A depicts the model for recovery of donor emission after photobleaching of an acceptor.

FIG. 7B is a series of emission images pre and post-photobleaching of AS-Alexa 568 and 647.

FIG. 8 depicts the results of an in vitro RNAi cleavage assay performed with double-stranded and single-stranded siRNA in the absence or presence of various concentrations of ATPA-18.

FIG. 9 is a schematic illustrating a method of synthesizing trifunctionalized ATPA-18K.

FIG. 10 is a schematic illustrating a method of synthesizing trifunctionalized compounds.

### DETAILED DESCRIPTION OF THE INVENTION

The present invention pertains to novel compounds, and methods of using these compounds to modulate RNAi. These novel compounds address difficulties in effectively mediating RNAi. Such compounds can be administered to a cell or organism to modulate the RNAi pathway. Optionally, one or more RNAi modulating compounds can be administered with an RNAi agent (e.g., siRNA, siRNA complexes and/or siRNA expression vectors) directed against one or more target RNAs. The invention further pertains to pharmaceutical compositions for treatment of diseases and disorders, and to kits including the RNAi modulating compounds of the invention.

For convenience, certain terms used in the specification, examples, and appended claims are collected here and throughout the application.

As used herein, the term “RNA interference” (“RNAi”) refers to a selective intracellular degradation of RNA. RNAi can occur in cells naturally to remove foreign RNAs (e.g., viral RNAs). RNAi proceeds via fragments cleaved from free dsRNA which direct the degradative mechanism to other similar RNA sequences. RNAi can be initiated by the hand of man, for example, to silence the expression of target genes.

The term “RNAi agent” as used herein, refers to an RNA (or analog thereof), comprising a sequence having sufficient complimentarity to a target RNA (i.e., the RNA being degraded) to modulate RNAi. A sequence having “sufficient complementarity to a target RNA sequence to direct RNAi” means that the RNAi agent has a sequence sufficient to trigger the destruction of the target RNA by the RNAi machinery (e.g., the RISC complex) or process. RNAi agents but are not limited to double stranded siRNA, single stranded siRNA (sense and/or antisense), shRNA and stRNA, modified and unmodified.

The terms “activate RNAi,” “activating RNAi,” “RNAi activation,” and the like refer to the enhancement of RNA interference (RNAi) by a compound of the invention. The enhancing activity of a compound can be determined by any means suitable for detecting RNAi activity in the presence of the compound. For example, enhancing activity can be determined by measuring an indicator of RNAi against a suitable control.

The terms “inhibit RNAi,” “inhibiting RNAi,” “RNAi inhibition,” and the like refer to the inhibition of RNAi by a compound of the invention. The inhibiting activity of a compound can be determined by any means suitable for detecting RNAi activity in the presence of the compound. For example, inhibiting activity can be determined by measuring an indicator of RNAi against a suitable control.

The terms “modulate RNAi” or “modulating RNAi” refer generally to the acts of enhancing and inhibiting RNAi. Exemplary compounds that modulate RNAi include ATPA18 and ATPA21, which inhibit RNAi, and ATPA2, ATPA12, ATPA20 and ATPA 24, which enhance RNAi.

As used herein, the phrase “indicator of RNAi” refers to any detectable marker, readout, etc. which is indicative of RNAi activity or an RNAi process occurring in said cell or organism. Levels of substrates or products of an RNAi process are preferred indicators. For example, levels (e.g., increasing levels) of siRNA-like molecules are indicative of RNAi. In another embodiment, levels of intermediate products (e.g., small duplex RNA) are indicative of RNAi. Other preferred indicators include levels of target RNA (e.g., target mRNA) and/or levels of protein encoded by a target mRNA. The latter, for example, can be indicative of target cleavage and/or translational repression. In certain embodiments, one or more substrate, product, intermediate, etc. is labeled (e.g., enzymatically, fluorescently or radioisotopically labeled to facilitate detection). Enzymatically labeled reagents are often assayed in the presence of a variety of colorimetric substances. Indirect assays, e.g., reporter gene assays sensitive to levels of proteins encoded by target mRNAs, are also suitable as indicators of RNAi. In preferred embodiments, a system as described above can further comprise suitable controls.

Various methodologies of the instant invention include a step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing a compound of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

A gene “involved” in a disorder includes a gene, the normal or aberrant expression or function of which effects or causes a disease or disorder or at least one symptom of said disease or disorder. Such disorders include viral infections (e.g., HIV and hepatitis), proliferative disorders (e.g., cancer), and regulatory disorders (e.g., related to aberrant expression of enzymes or factors in cascades such as a blood coagulation cascade).

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

The term “RNA” or “RNA molecule” or “ribonucleic acid molecule” refers to a polymer of ribonucleotides. The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). The single-stranded DNA and RNA can be the antisense strand or the sense strand (e.g., sense or antisense ss-RNA). The single or double stranded RNA can be chemically modified and/or crosslinked (e.g., RNA crosslinked with functionalized compounds such as psoralen and thio-uracil as disclosed herein). RNA also includes RNA linked with molecules such as dye molecules, and/or delivery vehicles such as Tat peptides, nanoparticles, etc. RNAi may be mediated against any of the RNA described herein using the methods of the invention.

The term RNA includes noncoding (“ncRNAs”) and coding RNAs (i.e., mRNAs). “mRNA” or “messenger RNA” is single-stranded RNA that specifies the amino acid sequence of one or more polypeptide chains. This information is translated during protein synthesis when ribosomes bind to the mRNA. ncRNAs are single- or double-stranded RNAs that do not specify the amino acid sequence of polypeptides (i e., do not encode polypeptides). By contrast, ncRNAs affect processes including, but not limited to, transcription, gene silencing, replication, RNA processing, RNA modification, RNA stability, mRNA translation, protein stability, and/or protein translation. ncRNAs include, but are not limited to, bacterial small RNAs (“sRNA”), microRNAs (“miRNAs”), and/or small temporal RNAs (“stRNAs”). The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference.

The term “oligonucleotide” refers to a short polymer of nucleotides and/or nucleotide analogs. The term “RNA analog” refers to a polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. The nucleotides of an analog may be linked with linkages that result in a lower rate of hydrolysis of the RNA analog as compared to an RNA molecule with phosphodiester linkages. For example, the nucleotides of an analog may comprise methylenediol, ethylene diol, oxymethylthio, oxyethylthio, oxycarbonyloxy, phosphorodiamidate, phophoroamidate, and/or phosphorothioate linkages. Exemplary RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA in that it has the ability to mediate (mediates) RNA interference.

As used herein, the term “isolated” molecule (e.g., isolated nucleic acid molecule) refers to molecules which are substantially free of other cellular material, or culture medium when produced by recombinant techniques, or substantially free of chemical precursors or other chemicals when chemically synthesized.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In certain embodiments, a straight chain or branched chain alkyl has 6 or fewer carbon atoms in its backbone (e.g., C₁₋₆ for straight chain, C₃₋₆ for branched chain). Likewise, preferred cycloalkyls have from 3-8 carbon atoms in their ring structure, and in certain embodiments preferably have 5 or 7 carbons in the ring structure. The term C₁₋₆ includes alkyl groups containing 1, 2, 3, 4, 5, or 6 carbon atoms. Optionally, the alkyl also may be interrupted by one or more O, NR^{a} or S groups, where R^{a} can be H, C₁₋₆alkyl, or C₁₋₆ acyl. In preferred embodiments, R^{a} is H.

Moreover, unless otherwise specified, the term alkyl may include both “unsubstituted” alkyls and “substituted” alkyls, the latter of which refers to moieties having substituents replacing at least one hydrogen on one or more carbons of the hydrocarbon backbone. Such substituents may include, for example, alkenyl, alkynyl, cycloalkyls, heterocycloalkyls, halogen (or halo), oxygen, including but not limited to hydroxyl (—OH) and oxo (═O) groups, sulfur or thio groups, including but not limited to thione (═S) and thiol (—SH), alkylcarbonyloxy, arylcarbonyloxy, alkoxy, alkoxyaryl, alkoxycarbonyl, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, carboxyl, alkylcarboxyl, phosphate, phosphonato, phosphinato, cyano, amino (including alkyl amino, dialkylamino, arylamino, aminoaryl, aminoheteroaryl, diarylamino, and alkylarylamino), acylamino (including alkylcarbonylamino, arylcarbonylamino, carbamoyl and ureido), amidino, imino, indole, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, aryl, heteroaryl, alkylaryl, a fused ring moiety, or an aromatic or heteroaromatic moiety. Cycloalkyls may also be further substituted, e.g., with the substituents described above. Those cycloalkyl groups having heteroatoms (e.g., O, N, NR^{a}, and S) in the ring structure may also be referred to as “cycloalkyl heterocycles,” or “heterocycloalkyls.” An “alkylaryl” or an “arylalkyl” moiety is an alkyl substituted with an aryl (e.g., phenylmethyl (i.e., benzyl)).

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. Likewise, cycloalkenyl groups may have from 3-8 carbon atoms in their ring structure, and more preferably have 5 or 7 carbons in the ring structure. The alkenyls also may be further substituted, e.g., with the substituents described above. The alkenyl also may be interrupted by one or more O, NR^{a} or S groups, where R^{a} can be H, C₁₋₆alkyl, or C₁₋₆ acyl. In preferred embodiments, R^{a} is H.

The term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. In certain embodiments, a straight chain or branched chain alkynyl group has 6 or fewer carbon atoms in its backbone (e.g., C₂₋₆ for straight chain, C₃₋₆ for branched chain). The alkynyls also may be further substituted, e.g., with the substituents described above. The alkynyl also may be interrupted by one or more O, NR^{a} or S groups, where R^{a} can be H, C₁₋₆alkyl, or C₁₋₆ acyl. In preferred embodiments, R^{a} is H.

In general, the term “aryl” includes groups, including 3- to 8-membered single-ring aromatic groups, that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. “Aryl” therefore includes both heteroaromatic and non-heteroaromatic moieties, unless otherwise indicated.

Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles,” “heterocycles,” “heteroaryls,” or “heteroaromatics”. Aryl groups may also be fused or bridged with alicyclic or heterocyclic rings that are not aromatic so as to form a polycycle (e.g., tetralin). The term aryl also can include aryls substituted, e.g., with the substituents described above.

The term “amine” or “amino” includes compounds or moieties in which a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl,” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “hydroxy” or “hydroxyl” includes groups with an —OH or —O⁻.

The term “halogen” or “halo” includes fluorine, bromine, chlorine, iodine, etc. The term “perhalogenated” generally refers to a moiety wherein all hydrogens are replaced by halogen atoms.

The term “heteroatom” includes atoms of any element other than carbon or hydrogen. Preferred heteroatoms are nitrogen, oxygen, sulfur, and phosphorus.

The term “fused ring moiety” or “fused ring system” refers to a ringed or multiringed structure, comprising one or more 3- to 8-membered cycloalkyl, heterocycloalkyl, aryl, or heteroaryl rings, which shares at least two adjacent carbons with an adjoining cycloalkyl, heterocycloalkyl, aryl or heteroaryl ring. Examples of fused ring moieties include, but are not limited to, indole, tetrahydropyridoindole, carbazole, indene, and dihydroindene. The rings also may be further substituted, e.g., with the substituents described above and/or may be interrupted by one or more O, NR^{a} or S groups, where R^{a} can be H, C₁₋₆ alkyl, or C₁₋₆ acyl. In preferred embodiments, R^{a} is H.

### I. RNAi Modulating Compounds

RNAi, the process by which RNA is targeted for degradation by an RNAi agent (e.g., a 21-23 nucleotide siRNA), is a natural mechanism that protects organisms against the proliferation of transposable elements in their cells that replicate via RNA intermediates. RNAi also can be employed to turn off expression or modulate over-expression of individual cellular genes. Once an RNAi agent (e.g., siRNA) is incorporated into an RNA-induced silencing complex (RISC), it is directed to RNA molecules, both single and double stranded, having complementary sequences for degradation. Thus, e.g., RNAi can be used to degrade single-stranded RNA (e.g., mRNA), double-stranded RNA (e.g., retroviral RNA such as HIV), and retroviral-like transposons. A RISC can operate a multiple of times without degradation and cellular mechanisms can also act to replicate degraded RNA to amplify RNAi. Moreover, RNAi can be spread from cell to cell by transfer of RNA fragments, and spread to progeny cells.

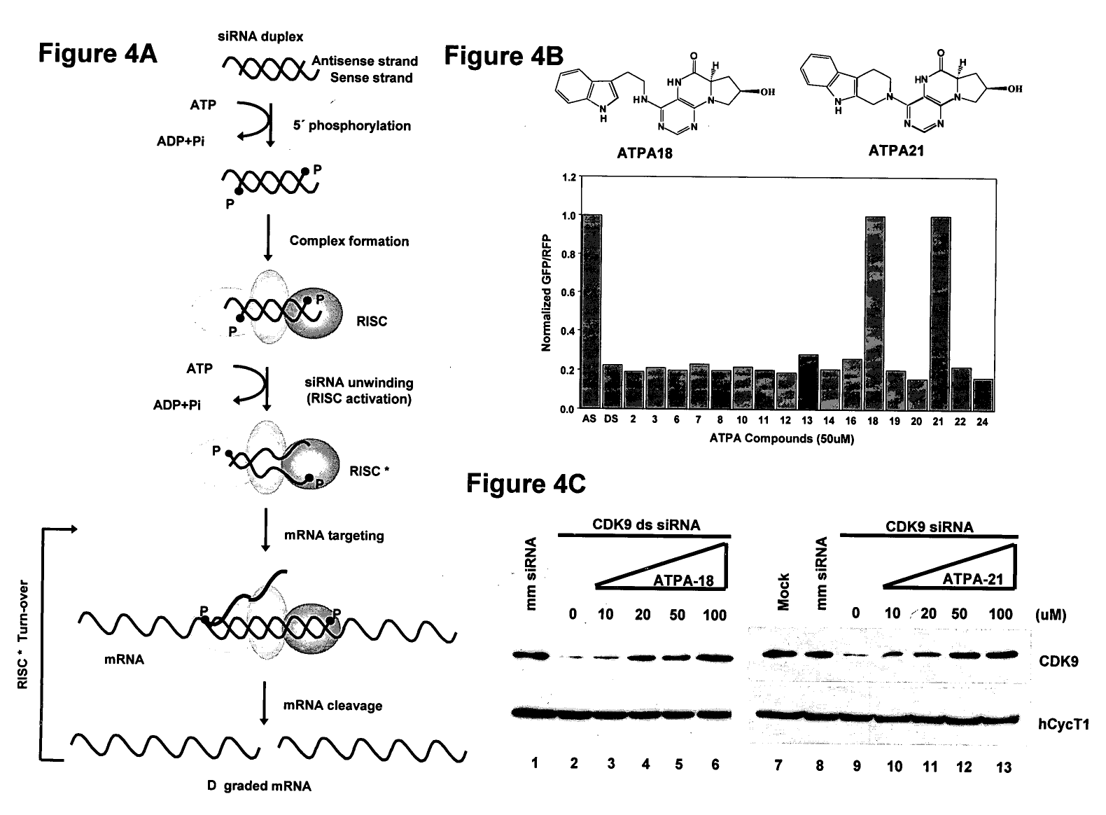

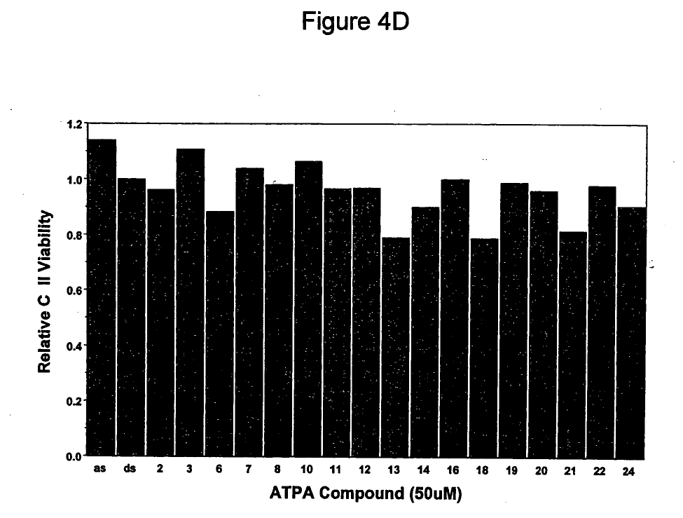

RNAi can be used to establish connections between gene structure and function in human cells by reverse genetics. S. M. Hammond et al., Nat Rev Genet 2, 110-9. (2001), M. T. McManus, P. A. Sharp, Nat Rev Genet 3, 737-747. (2002); M. Scherr, M. A. Morgan, M. Eder, Curr Med Chem 10, 245-56 (February, 2003). Because of its broad utility, understanding the mechanistic details behind the RNAi phenomenon is of great importance. A current model for how RNAi occurs in vivo is shown in FIG. 4A. In the first step of RNAi induction, the 5′ ends of the siRNA duplex are phosphorylated, resulting in formation of a RNA-induced silencing complex (RISC)-siRNA complex. Y. L. Chiu, T. M. Rana, Mol Cell 10, 549-561. (2002); S. M. Hammond et al., Nature 404, 293-6 (2000). Additional ATP-dependent events take place next, involving siRNA unwinding from the 5′ end of the antisense strand and activation of RISC (RISC*). A. Nykanen et al., Cell 107, 309-21. (2001). After RISC activation, the antisense strand of the unwound siRNA guides the siRNA-RISC* complex to the target mRNA. S. M. Hammond et al., Nature 404, 293-6 (2000). The guide antisense strand base pairs with the target mRNA, forming an A-form helical geometry recognized by RISC*. Y. L. Chiu, T. M. Rana, Mol Cell 10, 549-561. (2002). In the final step, target mRNA is cleaved by RISC* (S. M. Elbashir et al., Embo J. 20, 6877-88. (2001)), which is then recycled to cleave another mRNA.

While not wishing to be bound to any particular theory, it is believed that RNA is cleaved by Dicer in an ATP-dependent manner, and that the presence of compounds related to ATP can affect the RNAi pathway. As demonstrated herein, it is believed that the compounds modulate RNA helicase activity required for RNAi. Additionally or alternatively, the compounds of the invention may enhance or inhibit RNAi by sensitizing or de-sensitizing other molecules involved in the RNAi pathway. The compounds of the present invention also may stabilize or de-stabilize complexes or compounds in the RNAi pathway, such as RISC. The compounds of the invention also may participate in amplification of siRNAs or other RNAi agent, e.g., by promoting siRNA replication or multiple turnover events by RISC.

It is understood that the role of RNAi in mammalian cells, for example, humans, can also influence any one or more of the following phenomena such as the control of transposon activity, genetic instability (e.g., gene jumping), acquisition of chromosomal mutations, and DNA methylation. Accordingly, the compounds of the invention are also suitable for modulating such activities.

In particular, RNAi activity, typically comprising, for example, Argonaute, Dicer, and RNA-directed polymerase, or homologs thereof, has been observed to mediate gene silencing. This silencing can be in the form of reduced transposon activity, that is, the degree to which genetic elements, for example, repetitive elements such as transposon or retrotransposons (or even retroviruses, e.g., endogenous retroviruses which comprise repetitive elements), can leave the genome and/or integrate in other regions of a given genome. Because such genetic elements are frequently found in most eukaryotic genomes, the degree to which these elements move about the genome can influence, for example, the expression of nearby genes, gene regulatory networks, gene expression patterns in response to a cellular signal(s), or the acquisition of genetic lesions, e.g., chromosomal mutations. Moreover, it has been observed that altered DNA methylation can result in altered gene activity in the form of, for example, altered gene expression or the altered mobility of genetic elements, e.g., transposon activity. Importantly, as altered gene expression and in particular, genetic instability, have been linked to a number of biological processes and pathologies, e.g., aging, cancer, etc., it is desirable to be able to modulate the underlying mechanism of such activity.

Accordingly, in another embodiment, the compounds of the invention have application in the modulation (e.g., activation, inhibition, or maintenance) of the RNAi machinery such that desirable levels of gene expression, gene element mobility, and/or DNA methylation are achieved.

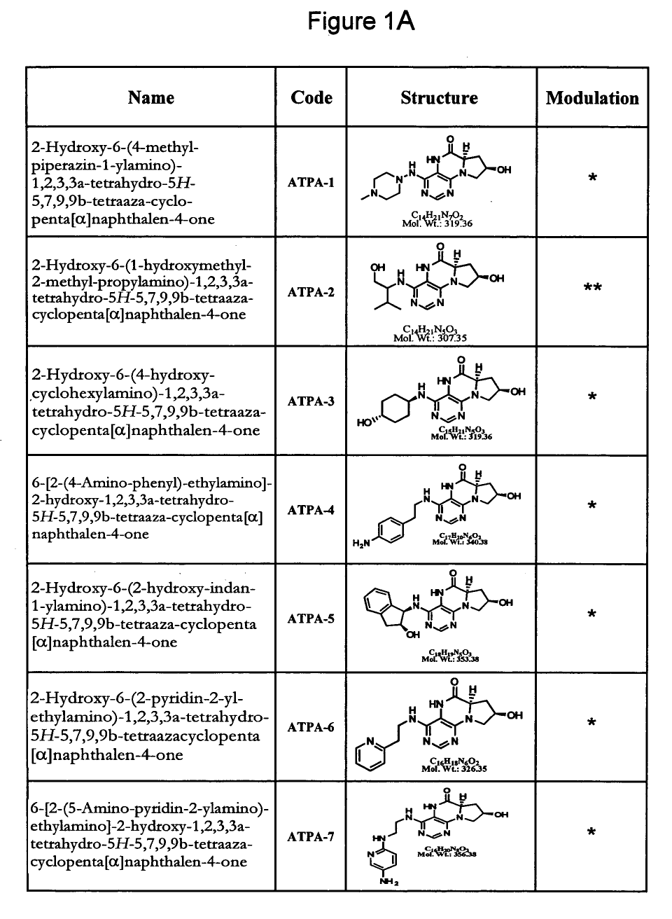

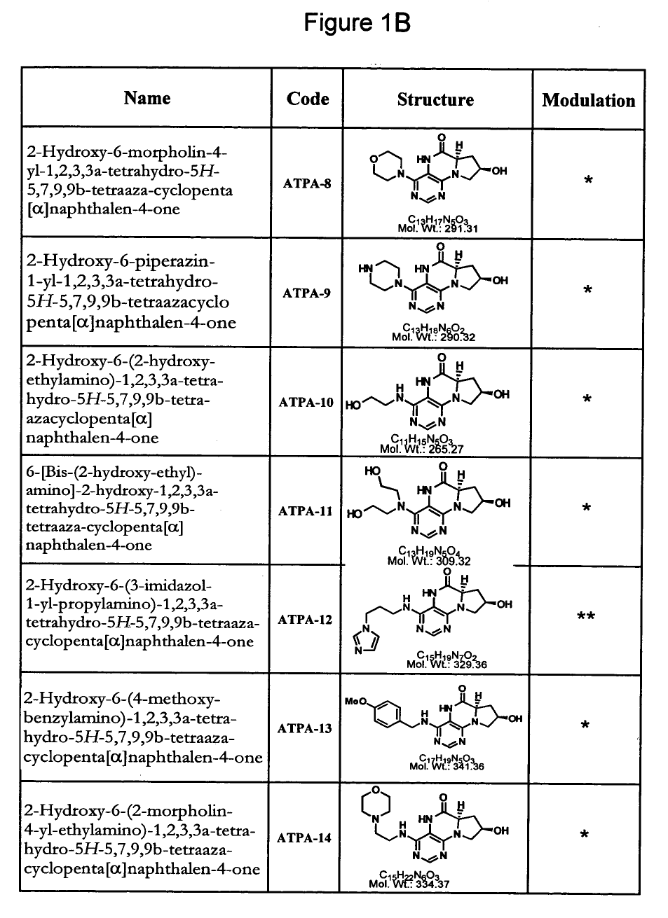

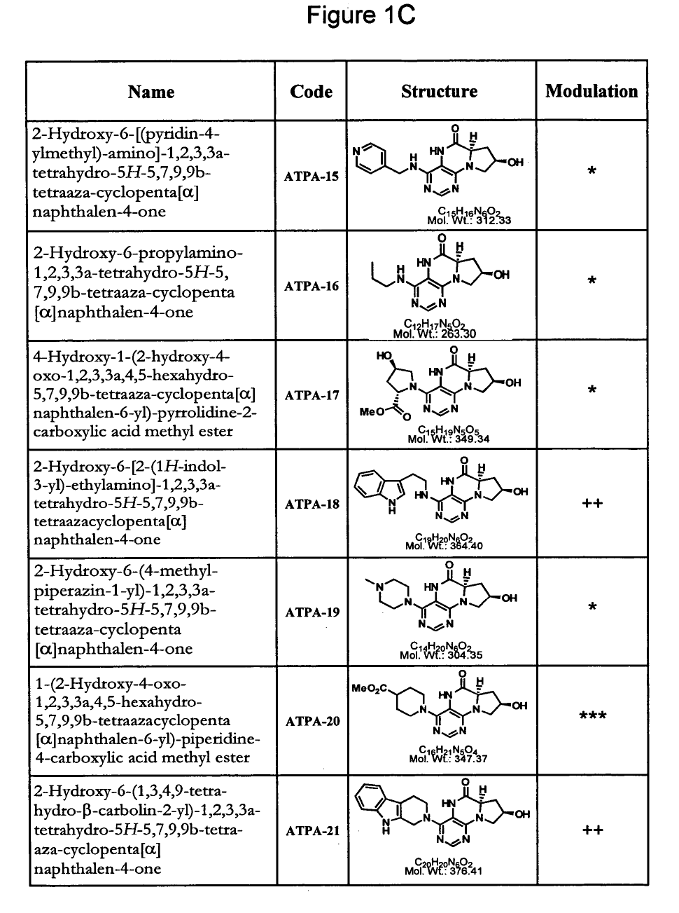

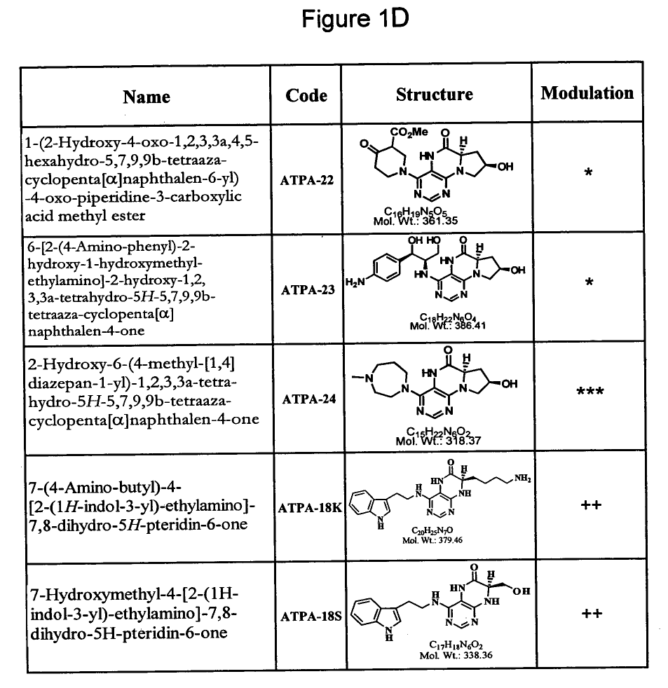

To study the ATP-dependent steps of the RNAi pathway, a novel chemical library of ATP analogs, depicted in FIG. 1A-D, was synthesized as described in detail below. Representing a novel approach to synthesizing a chemical library, the ATP-analog library was created by altering ATP molecules to have a more rigid scaffolding structure, making them less flexible. Some of the ATP analogs also were heterocyclic, having rings in addition to the adenine ring.

Using the dual fluorescence reporter assay described herein, the ATP analog (ATPA) compounds were screened for RNAi modulation. The results of the screen are shown in FIG. 4B and indicated in FIGS. 1A-D.

Two of the compounds, identified by structures and name under the designations ATPA-18 and ATPA-21 in FIG. 1C, showed complete inhibition of RNAi. ATPA-18 and ATPA-21 have similar structural features. For example, both have an indole structure attached to the base structure (Formula I), one fused more rigidly to a 6-membered ring, and one less rigidly attached to the base structure by an alkyl group.

Four of the compounds, identified by structures and name under the designations ATPA-2, ATPA-12, ATPA-20, and ATPA-24 in FIG. 1A-D, showed activation or enhancement of RNAi. Activators ATPA-20 and ATPA-24 showed better activation than ATPA-2 and ATPA-12, and have similar structural features. For example, both have a substituted heterocyclic structure attached to the base structure (Formula I). The screened compounds also were also non-toxic to cells as is shown in FIG. 4D which depicts the cell viability data for the compounds in FIG. 4C.

It should be noted that the RNAi modulating compounds of the invention are not limited to the modulators detected using the assay described herein. Any of the compounds described or depicted herein can be RNAi modulators, provided they are detected as modulators of RNAi. That is, further compounds may be identified as modulators using different assays or even using the same assay under different conditions (e.g., using different cells, concentrations of compound, buffers, and/or RNAi agents). Any compound described or depicted herein, including but not limited to those described in FIGS. 1A-D, that modulates RNAi is considered an RNAi modulating compound of the invention.

Accordingly, in one aspect, the present invention features compounds for modulating (enhancing or inhibiting) RNAi. The invention provides compounds for modulating RNAi, having the formula:

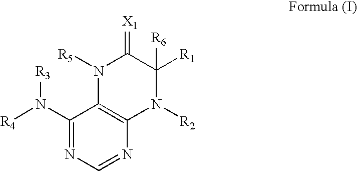

wherein

R¹ is alkyl, alkenyl, or alkynyl, optionally interrupted by one or more O, N, NR^{a}, or S groups, and optionally substituted with one or more hydroxyl, halo, alkoxy, oxo, amino, cycloalkyl, heterocycloakyl, aryl, heteroaryl, thione or thiol moiety, the cycloalkyl, heterocycloalkyl, aryl, or heteroaryl is optionally substituted with one or more oxo, hydroxyl, thione, thiol or fused ring moiety;

R¹ is H or C₁₋₆ alkyl, or R¹ and R² together form a 3- to 8-membered ring optionally interrupted by one or more O, NR^{a}, or S and optionally substituted with one or more hydroxyl, halo, alkoxy, oxo, amino, thione or thiol moiety;

R₃ is alkyl, alkenyl, alkynyl, aryl, cycloalkyl, or heterocycloalkyl, optionally interrupted by one or more O, NR^{a}, or S group, and optionally substituted with one or more hydroxyl, alkoxy, halo, aryl, heteroaryl, cycloalkyl, heterocycloalkyl, carboxyl, alkylcarboxy, oxo, amino, aminoaryl, aminoheteroaryl, indole, alkoxyaryl, alkoxycarbonyl, thione, thiol, or a fused ring moiety; and

R₄ is H or C₁₋₆ alkyl optionally substituted with a hydroxyl, halo, or amino group, or R³ and R⁴ together form a 3- to 8-membered ring optionally interrupted by one or more O, NR^{a}, or S and optionally substituted with one or more alkyl, hydroxyl, alkoxy, halo, aryl, heteroaryl, cycloalkyl, heterocycloalkyl, carboxyl, alkylcarboxy, oxo, amino, indole, thione, thiol or a fused ring moiety;

R⁵ is H or C₁₋₆ alkyl;

R⁶ is H or C₁₋₆ alkyl;

X¹ is O, S or NR^{a}; and

R¹ is H, C₁₋₆ alkyl, or C₁₋₆ acyl,

or a salt thereof.

In another aspect, the invention provides compounds for modulating RNAi, having the formula:

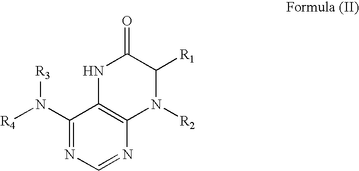

wherein

R¹ is alkyl, alkenyl, or alkynyl, optionally interrupted by one or more O, N, NR^{a}, or S groups, and optionally substituted with one or more hydroxyl, halo, alkoxy, oxo, amino, cycloalkyl, heterocycloalkyl, aryl, heteroaryl, thione or thiol moiety, the cycloalkyl, heterocycloalkyl, aryl, or heteroaryl optionally substituted with one or more oxo, thione or fused ring moiety;

R² is H or C₁₋₆ alkyl, or R¹ and R² together form a 3- to 8-membered ring optionally interrupted by one or more O, NR^{a}, or S and optionally substituted with one or more hydroxyl, halo, alkoxy, oxo, amino, thione or thiol moiety;

R₃ is alkyl, alkenyl, alkynyl, aryl, cycloalkyl, or heterocycloalkyl, optionally interrupted by one or more O, NR^{a}, or S group, and optionally substituted with one or more hydroxyl, alkoxy, halo, aryl, heteroaryl, cycloalkyl, heterocycloalkyl, carboxyl, alkylcarboxy, oxo, amino, aminoaryl, aminoheteroaryl, indole, alkoxyaryl, alkoxycarbonyl, thione, thiol, or a fused ring moiety;

R₄ is H or C₁₋₆ alkyl optionally substituted with a hydroxyl, halo, or amino group, or R³ and R⁴ together form a 3- to 8-membered ring optionally interrupted by one or more O, NR^{a}, or S and optionally substituted with one or more alkyl, hydroxyl, alkoxy, halo, aryl, heteroaryl, cycloalkyl, heterocycloalkyl, carboxyl, alkylcarboxy, oxo, amino, indole, thione, thiol or a fused ring moiety; and

R^{a} is H, C₁₋₆ alkyl, or C₁₋₆ acyl,

or a salt thereof.

In a preferred embodiment, R¹ is alkyl, wherein R¹ is optionally interrupted by one or more O or NH groups, and R¹ is optionally substituted with one or more hydroxyl, oxo, amino, cycloalkyl, heterocycloalkyl, aryl, or heteroaryl moiety, the cycloalkyl, heterocycloalkyl, aryl, or heteroaryl optionally substituted with one or more oxo, thione or fused ring moiety; R² is H, or R¹ and R² together form a 5- to 7-membered ring optionally substituted with one or more hydroxyl or amino moiety. Additionally or alternatively, in another preferred embodiment R₃ is alkyl, cycloalkyl, or heterocycloalkyl, optionally interrupted by one or more NH group, and optionally substituted with one or more hydroxyl, aryl, heteroaryl, cycloalkyl, heterocycloalkyl, amino, aminoaryl, aminoheteroaryl, indole, alkoxyaryl, or a fused ring moiety; R₄ is H or C₁₋₆ alkyl optionally substituted with a hydroxyl group; or R³ and R⁴ together form a 5- to 7-membered ring optionally interrupted by one or more O or NH, and optionally substituted with one or more alkyl, hydroxyl, alkoxy, carboxyl, alkylcarboxy, oxo, indole, or a fused ring moiety; and R^{a} is H or C₁₋₆ alkyl. Preferred embodiments also include salts of these compounds, more preferably a pharmaceutically acceptable salt.

In one embodiment, the RNAi modulating compound is an RNAi activator. In a particularly preferred embodiment, R₁ and R₂ together form a 3-hydropyrrolidine. In a second particularly preferred embodiment, the compound is selected from the group consisting of:

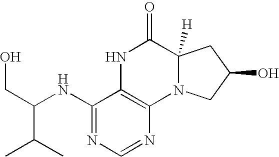

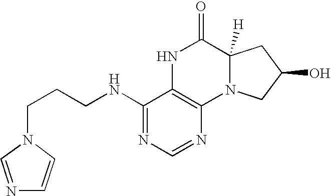

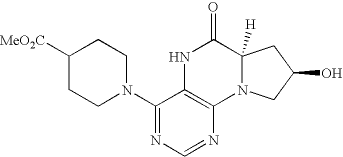

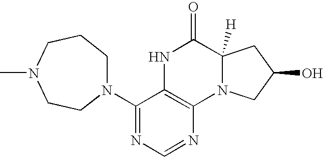

In another embodiment, the RNAi modulating compound is an RNAi inhibitor. In another particularly preferred embodiment, the compound is selected from the group consisting of:

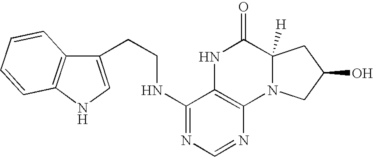

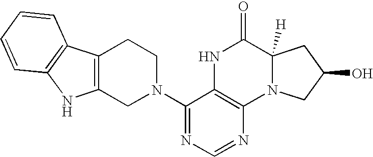

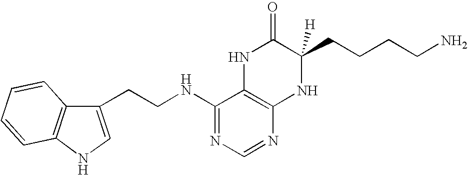

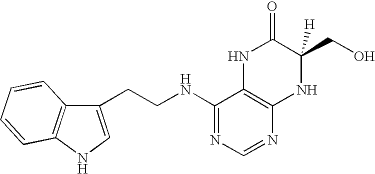

In one embodiment, the compound is a trifunctionalized compound. In a preferred embodiment, the compound is a trifunctionalized compound selected from the group consisting of:

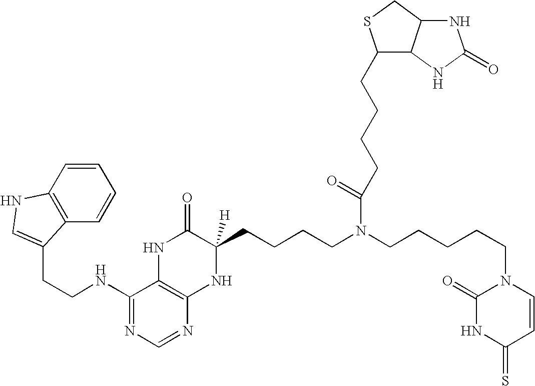

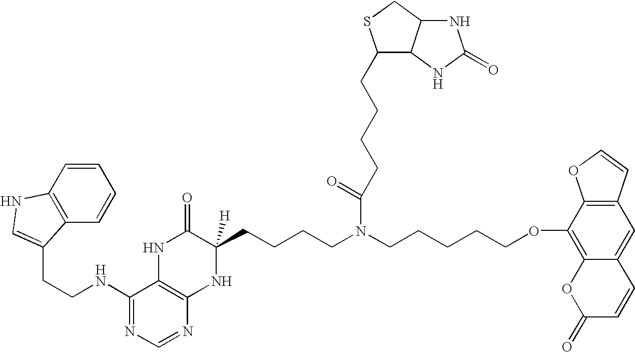

The above trifunctionalized compounds are trifunctionalized RNAi inhibitors, however, any of the compounds depicted or described herein, including but not limited to the compounds listed in FIG. 1A-D, can be trifunctionalized. These compounds can be used in any of the methods described herein.

Liquid Phase Synthesis of RNAi Modulating Compounds

The invention also includes a liquid-phase method of preparing a compound that includes the steps of contacting a 4,6-dihalo-5-nitro-pyrimidine with an alpha-amino ester to form a 4-amino-6-halo-5-nitro-pyrimidine; contacting the a 4-amino-6-halo-5-nitro-pyrimidine with a primary amine or a secondary amine to form a 4-amino-6-amino-5-nitro-pyrimidine; and reducing the 4-amino-6-amino-5-nitro-pyrimidine to form a dihydropteridinone.

The invention includes a liquid-phase method of synthesizing RNAi modulating compounds. In general, the compounds can be prepared by contacting a 4,6-dihalo-5-nitro-pyrimidine with an alpha-amino ester to form a 4-amino-6-halo-5-nitro-pyrimidine, contacting the a 4-amino-6-halo-5-nitro-pyrimidine with a primary amine or a secondary amine to form a 4-amino-6-amino-5-nitro-pyrimidine, and reducing the 4-amino-6-amino-5-nitro-pyrimidine to form a dihydropteridinone. More particularly, the compounds can be prepared according to Scheme I.

SCHEME I

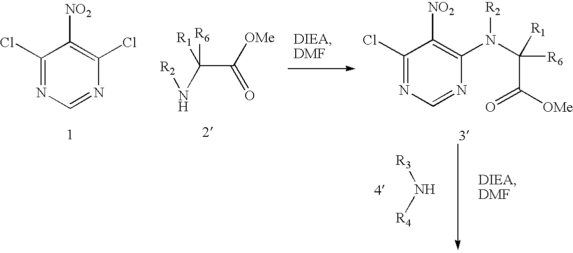

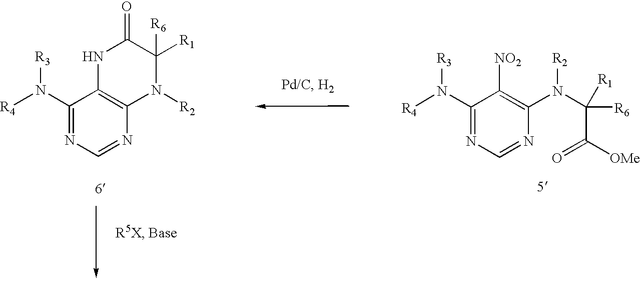

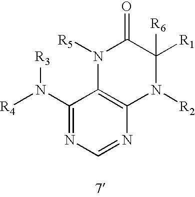

For example, in a first step, 4,6-dichloro-5-nitro-pyrimidine 1′ is combined with alpha- amino ester 2′ in the presence of a base, such as diisopropylethylamine (DIEA) to form compound 3′. In a second step, compound 3′ is combined with amine 4′ in the presence of DIEA to form compound 5′. In a third step, compound 5′ is subjected to hydrogenation conditions, such as catalytic palladium and a hydrogen atmosphere, to form dihydropteridinone 6′. Compounds in which R⁵ is not hydrogen can be prepared by mild alkylation to form compound 7′. Compounds in which X¹ is S can be prepared from compound 6′ according to procedures described in Sandier, S. R. and Karo, W., Organic Functional Group Preparations, Volume III (Academic Press, 1972) at pages 436-437. Where necessary, protecting groups can be used to facilitate synthesis of the compounds by the approach outlined in Scheme I. Protecting groups and reaction conditions can be found in T. W. Greene, Protective Groups in Organic Synthesis, (3rd, 1999, John Wiley & Sons, New York, N.Y.).

It is to be understood that the compounds of the invention can be further substituted, unsubstituted and otherwise derivatized provided such modification does not affect the ability of the compound to modulate RNAi. For example, further substituents can be added or replaced to adjust the hydrophobicity, hydrophilicity, or other properties of the compound to adjust or increase the activation or inhibition potential of the compound; these compounds fall within the scope of the present invention.

It will be noted that the structures of some of the compounds of this invention include stereogenic carbon atoms. It is understood accordingly that the isomers arising from such asymmetry (e.g., all enantiomers and diastereomers) are included within the scope of this invention, unless indicated otherwise. Such isomers may be obtained in substantially pure form by classical separation techniques and by stereochemically controlled synthesis. Furthermore, the structures and other compounds and moieties discussed in this application also include all tautomers thereof.

The invention also provides novel compositions, e.g., pharmaceutical compositions, including the RNAi modulating compounds of the present invention.

### II. RNAi Agents

The RNAi modulating compounds of the invention are capable of modulating (enhancing or inhibiting) both endogenous RNAi and RNAi mediated by addition of exogenous substrates. Exemplary exogenous substrates or RNAi agents include, e.g., double stranded siRNA duplexes, single stranded antisense RNA or single stranded sense RNA comprising a sequence having sufficient complementarity to at least one target mRNA sequence and/or expression vectors encoding at least one siRNA duplex or single stranded antisense or sense RNA comprising a sequence having sufficient complementarity to at least one target mRNA sequence.

siRNA duplexes, e.g., can be administered in an isolated state or can be manufactured by an siRNA expression vector introduced to the cell or organism. The siRNA molecule is preferably a duplex consisting of a sense strand and complementary antisense strand, the antisense strand having sufficient complementarity to mediate RNAi against a target RNA. Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA has a length from about 10-50 or more nucleotides, i.e., each strand comprises 10-50 nucleotides (or nucleotide analogs). More preferably, the siRNA has a length from about 15-45 nucleotides. Even more preferably, the siRNA has a length from about 18-25 nucleotides. The siRNA comprises a sequence having sufficient complementarity to a target mRNA sequence to direct target-specific RNA interference (RNAi). That is, the siRNA comprises a sequence having sufficient complementarity to trigger the destruction of the target mRNA by the RNAi machinery or process. In yet another embodiment, the RNAi agent is a single strand sense RNA, with or without modifications. In yet another embodiment the RNAi agent is a single strand antisense RNA.

The target RNA cleavage reaction guided by RNAi agents (e.g., by siRNAs) can be highly sequence specific. In general, siRNA containing or encoding nucleotide sequences identical to a portion of the target gene are preferred for inhibition. However, 100% sequence identity between the siRNA and the target gene is not required to practice the present invention. Sequence variations due, e.g., to genetic mutation, strain polymorphism, or evolutionary divergence, can be tolerated. For example, siRNA sequences with insertions, deletions, and single base mismatches relative to the target sequence have also been found to be effective for inhibition. Moreover, not all positions of a siRNA contribute equally to target recognition. Mismatches in the center of the siRNA are most critical and may essentially abolish target RNA cleavage. Mismatches upstream of the center or upstream of the cleavage site referencing the antisense strand are tolerated but significantly reduce target RNA cleavage. Mismatches downstream of the center or cleavage site referencing the antisense strand, preferably located near the 3′ end of the antisense strand, e.g., 1, 2, 3, 4, 5 or 6 nucleotides from the 3′ end of the antisense strand, are tolerated and reduce target RNA cleavage only slightly.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment is generated over only a portion of the sequence aligned (e.g., over a portion having significant identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

Alignment can be optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. Alignment also can be optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Greater than 90% sequence identity, e.g., 91%, 92%, 93%, 94%, 95%, 96%, 97%, 98%, 99% or even 100% sequence identity, between the siRNA and the portion of the target gene is preferred. Alternatively, the siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. Less than the melting temperature (TM) of the hybrid, where TM is determined according to the following equations. For hybrids less than 18 base pairs in length, TM(° C.)=2(# of A+T bases)+4(# of G+C bases). For hybrids between 18 and 49 base pairs in length, TM(° C.)=81.5+16.6(log10[Na+])+0.41(% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sam brook, J., E. F. Fritz, and T. Mania tis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 or 50 bases.

Additional or alternative RNAi agents that may be used in a method or composition of the invention, include a siRNA, wherein the strand has a sequence sufficiently complementary to a target RNA sequence to direct target-specific RNAi, and wherein the sense strand and/or antisense strand is modified by the substitution of internal nucleotides with modified nucleotides, such that in vivo stability is enhanced as compared to a corresponding unmodified siRNA. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In yet another embodiment, the modified nucleotides are present only in the antisense strand. In yet another embodiment, the modified nucleotides are present only in the sense strand. In yet other embodiments, the modified nucleotides are present in both the sense and antisense strand.

Preferred modified nucleotides or nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ moiety is a group selected from H, OR, R, halo, SH, SR, NH₂, NHR, NR₂ or ON, wherein R is C₁-C₆ alkyl, alkenyl or alkynyl and halo is F, Cl, Br or I.

Preferred are 2′-fluro, 2′-amino and/or 2′-thio modifications. Particularly preferred modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine; and/or 5-amino-allyl-uridine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides can be used within modified siRNAs of the instant invention, but are preferably included within the sense strand of the siRNA duplex. 2′-OMe nucleotides are less preferred. Additional modified residues have been described in the art and are commercially available but are less preferred for use in the modified siRNAs of the instant invention including, deoxy-abasic, inosine, N3-methyl-uridine, N6, N6-dimethyl-adenosine, pseudouridine, purine ribonucleoside and ribavirin. Modification of the linkage between nucleotides or nucleotide analogs is also preferred, e.g., substitution of phosphorothioate linkages for phosphodiester linkages.

Also possible are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable.

It should be noted that all modifications described herein may be combined.

The RNAi agents of the invention can further include crosslinked siRNA derivatives. Crosslinking can be employed to alter the pharmacokinetics of the composition, for example, to increase half-life in the body. Thus, e.g., an RNAi agent can include siRNA derivatives that include siRNA having two complementary strands of nucleic acid, such that the two strands are crosslinked. For example, a 3′ OH terminus of one of the strands can be modified, or the two strands can be crosslinked and modified at the 3′ OH terminus. The siRNA derivative can contain a single crosslink (e.g., a psoralen crosslink). In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying SiRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The RNAi agent can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al., Drug Deliv. Rev.:47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

The RNAi agents of the present invention can also be labeled using any method known in the art; for instance, the nucleic acid compositions can be labeled with a fluorophore, e.g., fluorescein, or rhodamine. The labeling can be carried out using a kit, e.g., the SILENCER™ siRNA labeling kit (Ambion). Additionally, the siRNA can be radiolabeled, e.g., using ³H, ³²p, or other appropriate isotope.

RNAi agents may be produced enzymatically or by partial/total organic synthesis, any modified ribonucleotide can be introduced by in vitro enzymatic or organic synthesis. siRNA can be prepared chemically: Methods of synthesizing RNA molecules are known in the art, in particular, the chemical synthesis methods as described in Verma and Eckstein (1998) Annul Rev. Biochem. 67:99-134. Alternatively, siRNA can be prepared enzymatically, e.g., a ds-siRNA can be prepared by enzymatic processing of a long ds RNA having sufficient complementarity to the desired target mRNA. Processing of long dsRNA can be accomplished in vitro, for example, using appropriate cellular lysates and ds-siRNAs can be subsequently purified by gel electrophoresis or gel filtration. ds-siRNA can then be denatured according to art-recognized methodologies. In an exemplary embodiment, RNA can be purified from a mixture by extraction with a solvent or resin, precipitation, electrophoresis, chromatography, or a combination thereof. Alternatively, the RNA may be used with no or a minimum of purification to avoid losses due to sample processing. Alternatively, the single-stranded RNAs can also be prepared by enzymatic transcription from synthetic DNA templates or from DNA plasmids isolated from recombinant bacteria. Typically, phage RNA polymerases are used such as T7, T3 or SP6 RNA polymerase (Milligan and Uhlenbeck (1989) Methods Enzymol. 180:51-62). The RNA may be dried for storage or dissolved in an aqueous solution. The solution may contain buffers or salts to inhibit annealing, and/or promote stabilization of the single strands. siRNA molecules of the instant invention may be modified, e.g., chemically modified, to increase a desired property of the molecule, e.g., the stability of the molecule or the activity of the molecule.

Compositions of the present invention can further include other molecules for mediating RNAi, such as elements of the RNA-induced silencing complex (RISC) and/or Dicer endonuclease.

RNAi agents can target a variety of genes, including those specifying the amino acid sequence of a cellular protein, a normal or mutated protein involved in a disorder, a protein involved in DNA repair/maintenance, or viral proteins, such as envelope proteins necessary for proliferation of the virus. As used herein, the phrase “specifies the amino acid sequence” of a protein means that the mRNA sequence is translated into the amino acid sequence according to the rules of the genetic code. siRNAs can be synthesized in vivo, in situ, or in vitro. Endogenous RNA polymerase of the cell may mediate transcription in vivo or in situ, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the siRNA. Inhibition may be targeted by specific transcription in an organ, tissue, or cell type; stimulation of an environmental condition (e.g., infection, stress, temperature, chemical inducers); and/or engineering transcription at a developmental stage or age.

A transgenic organism that expresses siRNA from a recombinant construct may be produced by introducing the construct into a zygote, an embryonic stem cell, or another multipotent cell derived from the appropriate organism.

### III. Modulating RNAi in a Cell or Organism

The present invention provides methods of modulating RNAi in a cell, organism, or a subject comprising the step of contacting the cell with, or administering to the organism or subject, a compound or compositions of the invention such that RNAi is modulated in the cell, organism or subject.

The compounds of the invention can be administered via a wide variety of administration methods including orally, by injection, lipid-mediated carrier transport, chemical-mediated transport, such as calcium phosphate, and the like. Methods of introducing small molecules into the body are well known and are discussed in greater detail below. Physical methods of introducing one or more substrates include injection of a solution containing the substrate, bombardment by particles covered by the substrate, soaking the cell or organism in a solution including the substrate, or electroporation of cell membranes in the presence of the substrate. A viral construct packaged into a viral particle also can accomplish both efficient introduction of an expression construct into the cell and transcription of RNA encoded by the expression construct.

Compounds may be directly introduced into the cell (i.e., intracellularly); or introduced extracellularly into a cavity, interstitial space, into the circulation of an organism, introduced orally, or may be introduced by bathing a cell or organism in a solution containing the compounds. Vascular or extravascular circulation, the blood or lymph system, and the cerebrospinal fluid are sites where the compounds may be introduced.

The cell with the target gene may be derived from or contained in any organism. The organism may a plant, animal, protozoan, bacterium, virus, or fungus. Examples of vertebrate animals include fish, mammal, cattle, goat, pig, sheep, rodent, hamster, mouse, rat, primate, and human; invertebrate animals include nematodes, other worms, drosophila, and other insects.

The cell having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Depending on the particular target gene and the dose of compounds and, optionally, siRNA and/or siRNA expression vectors delivered, this process may provide partial or complete loss of function for the target gene. A reduction or loss of gene expression in at least 50%, 60%, 70%, 80%, 90%, 95% or 99% or more of targeted cells is exemplary. Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

Accordingly, the present invention also provides knock-down or knock-out cells or organisms exhibiting a target-gene specific knock-out or knock-down phenotype that include a compound or composition of the invention that enhances RNAi. The cell or organism includes RNAi agent, which comprises a sequence having sufficient complementarity to a target gene to direct target-specific siRNA interference, and/or an siRNA expression vector capable of inhibiting expression of a target gene. Such knock-out and knock-down cells and organisms are described in greater detail below.

### IV. RNAi and RNAi Modulation Assays

A candidate compound is a compound whose ability to modulate (e.g., inhibit or activate) RNAi is unknown. Such a compound can be prepared according to the methods disclosed herein and can be tested for the ability to modulate (e.g., inhibit or activate) RNAi. Any system in which RNAi activity can be detected can be used to test the activity (e.g., inhibitory or activating activity) of a candidate compound. In general, a system in which RNAi activity can be detected is incubated in the presence and absence of a candidate compound. If RNAi activity is inhibited in the presence of the compound and is not inhibited in the absence of the compound, then the compound is an inhibitory compound (an RNAi inhibitor). If RNAi activity is enhanced in the presence of the compound and is not enhanced in the absence of the compound, then the compound is an activating compound (an RNAi activator).

In one aspect, the invention provides a method of identifying an RNAi modulator. The method includes the steps of (a) contacting a cell or cell extract comprising a target gene which encodes a target protein with a RNAi agent targeted against the target gene and a candidate compound; and (b) detecting a measure of the target protein in the cell or cell extract, wherein a variation in the measure of the target protein against a suitable control identifies the candidate compound as an RNAi modulator.

In a preferred embodiment, the candidate compound is an ATP analog. The ATP analog can be any of the ATP analogs described or depicted herein. In one embodiment, the target gene is a cellular gene. In another embodiment the target gene is a viral gene. In another embodiment the target gene is a reporter gene, e.g., a fluorescent reporter protein or polypeptide. The cell or cell extract can further include a reference gene encoding a reference protein or polypeptide, e.g., a fluorescent reference protein or polypeptide.

In a preferred embodiment, the measure of the reporter protein is a ratio of a level or activity of the reporter protein to a level or activity of a reference protein and/or the suitable control is a cell comprising the reporter gene contacted with an antisense molecule targeted against the reporter gene or an siRNA targeted against the reporter gene.

In the methods and systems of the present invention, the ability of a compound to modulate RNAi and/or the degree to which it modulates RNAi can be measured in a number of ways. In one embodiment, the level or activity of a target protein (a protein expressed by a target gene) can be measured as an indicator of RNAi activity. The level or activity of target protein generally can be divided by the level or activity of a reference protein (a protein expressed by a reference protein). It should be understood that RNAi activity may be measured by any indicator of RNAi and is not limited to the measure of target protein.

In one embodiment, the ability of a compound to modulate RNAi and/or the degree to which it modulates RNAi can be measured as an increase or decrease in target protein level (preferably divided by a reference protein level in each cell, cell extract or organism), relative to that observed without the candidate compound. If the system contacted with the candidate compound includes a RNAi agent (exogenous or endogenous), preferably, the system without the candidate compound also includes the RNAi agent. In certain embodiments, the RNAi activity can be normalized to that observed in the absence of both an RNAi agent and the compound (e.g., where 1 represents the level or activity of the target protein in the absence of RNAi, and 0 represents the target protein level where the gene is completely repressed by RNAi). Thus, if the normalized target protein level in a system without a candidate compound is 0.2 and the normalized target protein level in a system with a candidate compound is 0.1, the candidate compound can be said to activate RNAi by 50% (by reducing the protein level by half of original protein level). This measurement can be helpful when comparing the ability of a number of candidate compounds to modulate RNAi against the same target protein. Preferably, a candidate compound modulates (enhances or inhibits) RNAi by at least 2%, more preferably at least 5%, even more preferably at least 7%. The activator compounds of the invention can modulate (enhance or inhibit) RNAi by 2%, 10%, 15% . . . 90%, 97%, 100%, 200%, 500% . . . 1000%, including all individual values and intermediate ranges. The minimum activation necessary to consider a candidate compound a modulator can be as low or as high as appropriate to the application for the compound, from the smallest amount of modulation measurable by the particular assay to the highest amount including the endpoints.

In another embodiment, the ability of a candidate compound to modulate RNAi and/or the degree of RNAi modulation can be measured as a percentage increase or decrease in the normalized protein level relative to that observed in the absence of RNAi activity (wherein the level is normalized to that observed in the absence of compound and RNAi agent as described above). Thus, e.g., if a system without a candidate compound of the invention exhibits a protein level of 0.5 in the presence of an RNAi agent, and the system with a candidate compound of the invention exhibits a protein level of 0.3, then the candidate compound can be said to activate RNAi by 0.2 (or 20%). This measurement can be useful, e.g., when a measure of the overall change in RNAi activity relative to the two endpoints is desired. This embodiment can be used, e.g., when comparing the modulatory activity of a compound against two different target genes. Preferably, a candidate compound modulates (enhances or inhibits) RNAi by at least 2%, more preferably at least 5%, even more preferably at least 7%. The activator compounds of the invention can modulate (enhance or inhibit) RNAi by 2%, 10%, 15% . . . 90%, 97%, 100%, including all individual values and ranges within this range. The minimum activation necessary to consider a candidate compound a modulator can be as low or as high as appropriate to the application for the compound, from the smallest amount of modulation measurable by the particular assay to the highest amount including the endpoints.

In yet another alternative embodiment, the ability to modulate and the degree of modulation can be expressed in terms of degree of movement in RNAi activity to an endpoint. Thus, for example, the compound in the previous embodiment could be said to activate RNAi by 0.4 or 40% because it resulted in an increase of 40% of the total increase necessary to completely repress the target gene. This measurement can be used, e.g., when assessing a number of different dosages of compound and it is desirable to assess the movement towards an endpoint (i.e., total inhibition of RNAi or total repression of the target gene). This can be useful, e.g., in determining a dosage of compound that will not entirely shut off a gene or the RNAi pathway when doing so is detrimental to the system (e.g., toxic to a cell or an organism), yet some degree of modulation is necessary or desired. Preferably, a candidate compound modulates (enhances or inhibits) RNAi by at least 2%, more preferably at least 5%, even more preferably at least 7%. The activator compounds of the invention can modulate (enhance or inhibit) RNAi by 2%, 10%, 15% . . . 90%, 97%, 100%, including all individual values and ranges within this range. The minimum activation necessary to consider a candidate compound a modulator can be as low or as high as appropriate to the application for the compound, from the smallest amount of modulation measurable by the particular assay to the highest amount including the endpoints.

In a particularly preferred embodiment, the invention provides a dual fluorescence reporter gene assay (DFRG assay) that can be used to test candidate ATPA compounds. The DFRG assay can also be used to test the ability of other types of compounds to modulate (e.g., inhibit or activate RNAi).

In the DFRG assay, cells that have RNAi activity are tansfected with two reporter plasmids that encode and can express different proteins that can be detected by fluorescence (fluorescent reporter genes that encode fluorescent reporter proteins). The fluorescence emission spectra of the two proteins are such that they can be distinguished when expressed simultaneously, e.g., red fluorescent protein (RFP) and green fluorescent protein (GFP). The cells are also transfected with a siRNA targeted to one of the reporter genes. Methods of designing such siRNAs are known in the art. A control culture of the cells with the siRNA (i.e., a positive control culture, i.e., positive for RNAi) is incubated for a time sufficient to produce detectable reporter protein (in the absence of candidate compound, e.g., candidate RNAi inhibitor or activator). A test culture of transfected cells with the siRNA is incubated for the same amount of time in the presence of a candidate compound, candidate RNAi inhibitor or activator, e.g., a candidate ATPA). The candidate compound (e.g., candidate RNAi inhibitor or activator) can be added near the time of transfection or within about 6 hours before or after transfection.

After incubation, the cells are harvested and cleared lysates are prepared from each of the cultures (control lysate and test lysate). Fluorescence of the lysates is measured using methods known in the art. A comparison is made between the emission intensity in the test lysate and control lysate. In a preferred embodiment, the fluorescence intensity ratio of target fluorophore to control fluorophore is determined. In a more preferred embodiment, the fluorescence intensity ratio of target fluorophore to control fluorophore is determined and is further normalized, e.g., to a suitable control or reference.

In some embodiments of the invention, the suitable control or reference features a culture contacted with transfection reagents, absent any nucleic acid. In some embodiments of the invention, the suitable control or reference features a culture is transfected with an antisense strand siRNA. The latter culture is also referred to as a non-specific control based on the fact that any target degradation detected is presumed to occur via a non-RNAi-based mechanism. Normalized ratios of less than one (i.e., less fluorophore expression in the cell contacted with the targeted siRNA than in the non-specific control cell) indicate target sequence-specific interference (irrespective of the addition of candidate compound). Normalized ratios of 1 indicate lack of target sequence-specific interference due, for example, to inhibition of interference by the candidate compound (or possibly due to lack of substrate, i.e., targeted siRNA). Intermediate normalized ratios indicate varying degrees of inhibition of sequence-specific interference.

In yet other embodiments, suitable control or reference is the culture transfected with targeted siRNA, i.e., the fluorescence ratio from the test culture is normalized to that from the positive control culture. Normalized ratios of 1 indicate target sequence-specific interference. Normalized ratios of less than 1 indicate activation of interference by the candidate compound. Normalized ratios of greater than 1 indicate inhibition of interference by the candidate compound.

In some embodiments of the invention, proteins from the lysates are prepared as described above and analyzed using Western blotting. Briefly, the proteins prepared from the transfected cells (control cells and test cells) are subjected to SDS-PAGE (e.g., in a 10% gel) and transferred to a membrane suitable for Western blotting (for example, a PVDF membrane). The membrane is immunoblotted using methods known in the art to detect the fluorescent reporter proteins. In general, a protein that can be used as a control for protein loading (such as a housekeeping protein) is also detected.

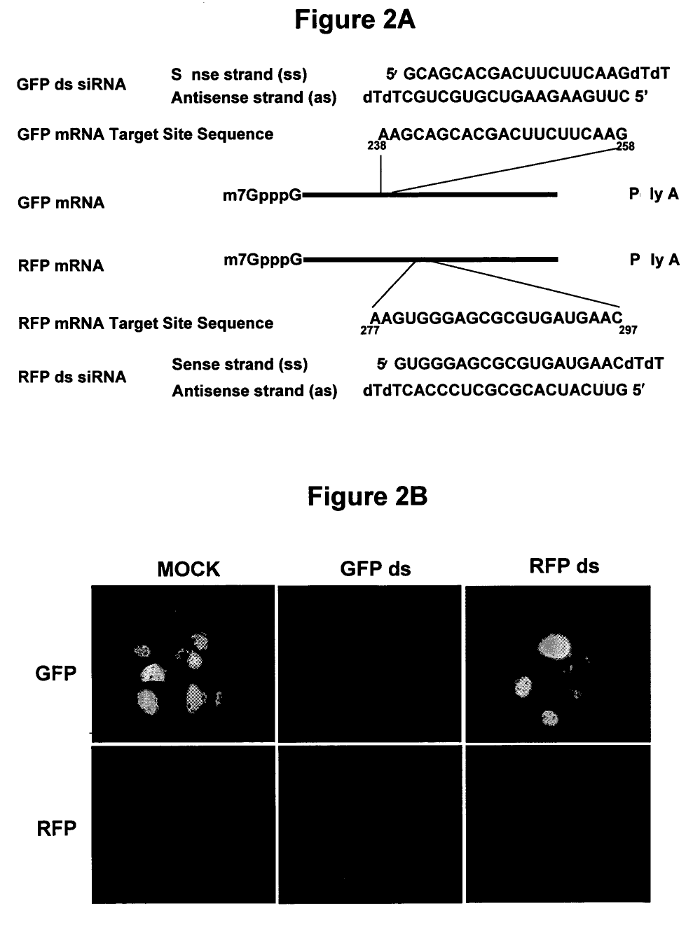

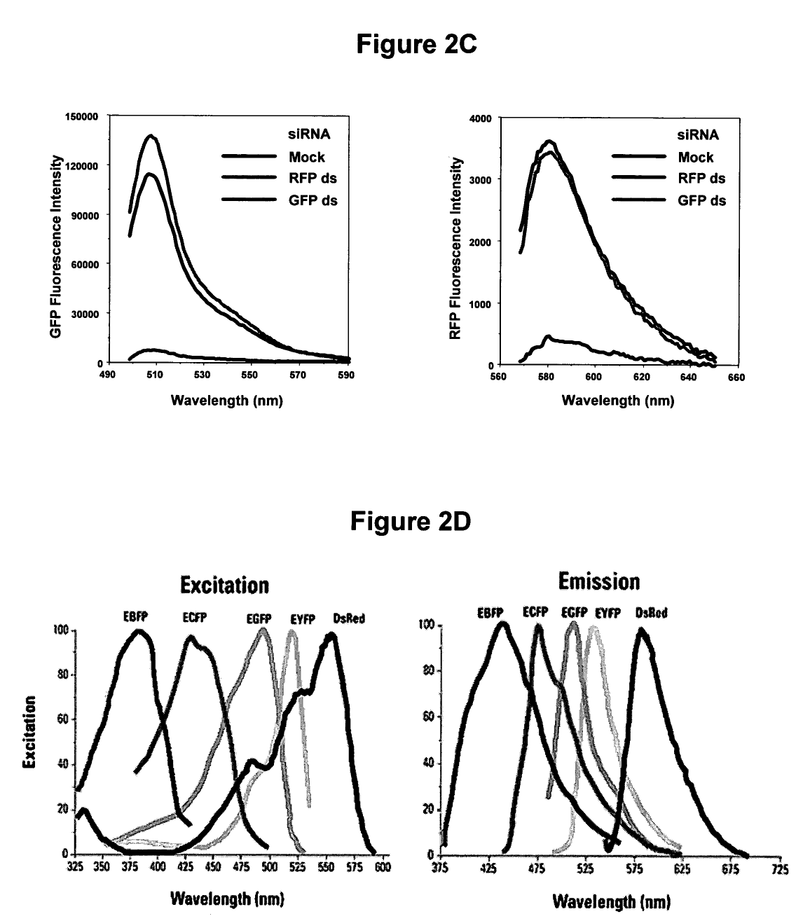

Cells to be used in a DFRG assay are generally cultured mammalian cells, e.g., human cells. The cells can be immortal, primary, or secondary cells. Cells from other organisms that exhibit RNAi or RNAi-type activity such as quelling can also be used. Such cells include those from fungi, plants, invertebrates (e.g., Drosophila melanogaster and Caenorhabditis elegans), vertebrates (e.g., zebrafish and mouse). Fluorescent molecules that can be used in DFRG assays are pairs of fluorescent molecules whose emission spectra can be distinguished when there is simultaneous emission. Examples of such pairs include Green Fluorescent Protein (GFP) and Red Fluorescent Protein (RFP). Additional examples can be selected, e.g., from those shown in Table 1 and FIG. 2D. In a preferred embodiment, EGFP-C1 (encoding enhanced GFP), and DsRed2-N1 (encoding RFP DsRed, a variant that has been engineered for faster maturation and lower non-specific aggregation) is used.

TABLE 1
LIVING COLORS FLUORESCENT PROTEINS

| Fluor. | Excit./Emiss. | Excitation | Quantum | |
|---|---|---|---|---|
| Protein | Maxima (nm) | Coefficient | Yield | Reference: |
| DsRed | 558/583 | 22,500 | 0.23 | Malz et al. 1999 |
| EGFP | 488/507 | 55,000 | 0.60 | D. W. Piston.- |
| EYFP | 513/527 | 84,000 | 0.61 | Vanderbilt |
| ECFP | 433/475 | 26,000 | 0.4 | University, |
| EBFP | 380/440 | 31,000 | 0.18 | Personal comm. |

Inducible gene expression systems (e.g., TET-OFF/TET ON System Dose Response, TET-OFF/TET-ON System Time Course available from Knoll GmbH (Germany) and other products that allow the control of target gene expression by hormones/steroids) can also be utilized in the assays of the present invention. Inducible gene expression systems are a means for controlling the timing and level of expression of genes in cells and organisms and can also be used, e.g., in gene therapy utilizing compounds of the present invention and knock-down or knock-out cells or organisms of the invention.

Further alternative reporter genes that can be used in the assays of the present invention include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of modulation which is greater than 7%, 10%, 33%, 50%, 90%, 95% or 99% as compared to a cell not treated according to the present invention. Lower doses of injected material and longer times after administration of compositions of the invention may result in modulation in a smaller fraction of cells (e.g., at least 10%, 20%, 50%, 75%, 90%, or 95% of targeted cells). Quantitation of gene expression in a cell may show similar amounts of modulation at the level of accumulation of target mRNA or translation of target protein. As an example, RNAi modulation may be determined by assessing the amount of gene product in the cell; mRNA may be detected with a hybridization probe having a nucleotide sequence outside the region used for the inhibitory double-stranded RNA, or translated polypeptide may be detected with an antibody raised against the polypeptide sequence of that region.

The modulation may be assessed by normalizing the activities relative to RNAi activity in the absence of the administration of the compounds or composition of the invention.

### V. Methods of Treatment

The present invention provides for both prophylactic and therapeutic methods of treating a subject at risk of (or susceptible to) a disorder or having a disorder associated with aberrant or unwanted target gene expression or activity, or a disease or disorder which can be treated or prevented by decreasing the amount of the target gene that is translated.

“Treatment”, or “treating” as used herein, is defined as the application or administration of a therapeutic agent (e.g., RNAi modulating compounds of the invention) to a patient, or application or administration of a therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease. For example, the methods of treatment of the instant invention provide for administration of RNAi modulation compounds, and optionally an RNAi agent specific for genes that are involved in the disease or disorder, such that RNAi against the target gene is enhanced or inhibited.

The methods of the present invention include methods of administering the compounds of the invention to attenuate RNAi at desired times. RNAi activating compounds can be adminstered to activate RNAi at desired times and RNAi inhibiting compounds can be administered to inhibit RNAi at desired times. For example, an RNAi activating compound or composition of the invention can be added to enhance RNAi to treat a disorder, such as a blood clotting disorder, when needed, e.g., during or after a trauma or surgery to attenuate production of a polypeptide involved in a cascade. Similarly, when RNAi activity is no longer necessary or desired, an RNAi inhibiting compound can be administered to reduce or eliminate RNAi activity.

The present invention provides a method for treating a subject that would benefit from administration of a compound of the present invention. Any therapeutic indication that would benefit from RNAi modulation can be treated by the methods of the invention. Accordingly, the present invention provides methods of treating a subject at risk for or having a disease or disorder associated with or related to normal or aberrant expression of a gene. The method includes the step of administering to the subject a compound or compositions of the invention, such that RNAi against the gene is modulated. The disease or disorder can be, e.g., a cellular growth or proliferative disorder, a viral infection, or a gene mutation disorder. The methods also can be used to target genes involved in pain, arthritis (TNF), ocular and/or macular degeneration, and venereal diseases (e.g., herpes, including topical herpes, and HPV). Further examples include mediation of RNAi against a target gene involved in skin disorders or diseases (e.g., a gene involved in the development, progression, etc. of psoriasis, and topical cancers).

As used herein, a “cellular growth or proliferation disorder” includes a disease or disorder that affects a cellular growth or proliferation process. As used herein, a “cellular growth or proliferation process” is a process by which a cell increases in number, size or content, by which a cell develops a specialized set of characteristics which differ from that of other cells, or by which a cell moves closer to or further from a particular location or stimulus. A cellular growth or proliferation process includes the metabolic processes of the cell and cellular transcriptional activation mechanisms. A cellular growth or proliferation disorder may be characterized by aberrantly regulated cell growth, proliferation, differentiation, or migration. Cellular growth or proliferation disorders include tumorigenic disease or disorders. As used herein, a “tumorigenic disease or disorder” includes a disease or disorder characterized by aberrantly regulated cell growth, proliferation, differentiation, adhesion, or migration, resulting in the production of or tendency to produce tumors. As used herein, a “tumor” includes a normal benign or malignant mass of tissue. Examples of cellular growth or proliferation disorders include, but are not limited to, cancer, e.g., carcinoma, sarcoma, or leukemia, examples of which include, but are not limited to, colon, ovarian, lung, breast, endometrial, uterine, hepatic, gastrointestinal, prostate, and brain cancer; tumorigenesis and metastasis; skeletal dysplasia; and hematopoietic and/or myeloproliferative disorders.

Treatment of cellular growth and proliferation disorders can include administering a compound of the invention (optionally in the present of an RNAi modulating agent or an expression vector encoding the same), such that aberrant cellular growth and proliferation is mediated. For example, RNAi can be activated that targets kinases critical to human cancer cells, vascular endothelial growth factor (VEGF), which has several functions in promoting tumor development, and/or signaling/repair proteins. Other potential targets include fatty acid synthases specific to malignant cells, and promatrix metalloproteinase-9 (MMP-9) associated with human tumor invasion and metastatis. Accordingly, the methods of the invention can also be used to treat a subject having a cell proliferation disorder such that the cell proliferation disorder does not metastasize. Yet another potential target is CDK9, which is multifunctional and linked to a number of disorders, including cell proliferation disorders, apoptosis, HIV transcription, hypertrophy (heart enlargement), immunology, tumorgenesis, and cancer.

As used herein, the term “viral infection” includes infections with organisms including, but not limited to, retroviruses including HIV (e.g., HIV-1 and HIV-2), herpes viruses, cytomegalovirus, Rotavirus, Epstein-Barr virus, Varicella Zoster Virus, hepatitis viruses (e.g., hepatitis B virus, hepatitis A virus, hepatitis C virus and hepatitis E virus), paramyxoviruses, Respiratory Syncytial virus, parainfluenza virus, measles virus, mumps virus, human papilloma viruses (for example HPV6, 11, 16, 18 and the like), flaviviruses (e.g. Yellow Fever Virus, Dengue Virus, Tick-borne encephalitis virus, Japanese Encephalitis Virus), coronaviruses (e.g., Severe Acute Respiratory Syndrome (SARS)), viral hemorrhagic fevers (VHFs) (e.g., Ebola hemorrhagic fever, Lassa fever, hantavirus pulmonary syndrome (HPS), and hemorrhagic fever with renal syndrome (HFRS)), small pox, filoviruses (e.g., Marburg hemorrhagic fever virus), arenaviruses (e.g., Lassa virus, Junin virus, Machupo virus, Guanarito virus, lymphocytic choriomeningitis virus (LCM and LCMV), and Sabia virus), paramyxo viruses (e.g., Hendra virus encephalitis and Nipah virus), Hanta virus, and phleboviruses (e.g., Rift Valley Fever (RVF) virus). Treatment of viral infections can include administering a compound of the invention (optionally in the presence of an RNAi modulating compound or an expression vector encoding the same), such that RNAi against viral RNA enhanced. Preferably, the treatment interferes with the replication of the virus, and more preferably, the treatment halts the replication of the virus.

A “gene mutation disorder” is a disorder or disease associated with or related to a gene mutation. Gene mutation disorders include gene mutations that render a protein ineffective of defective, which can interrupt enzyme action (e.g., thrombotic thrombocytopenic purpura (TTP) caused by mutations in a gene that render the ADAMTS13 enzyme ineffective), and gene mutations that cause disruptions in a cascade such as a coagulation cascade (e.g., hemophilia and related coagulation Factor defect disorders). Additional exemplary gene mutation disorders include Parkinson's Disease, Alzheimer's Disease, epilepsy, Charcot-Marie-Tooth disease (CMT), Huntington's disease (HD), ALS (Lou Gherig's Disease), and Muscular Distrophy (MD). Treatment of gene mutation disorders can include administering a compound of the invention (optionally in the present of an RNAi modulating agent or an expression vector encoding the same), such that RNAi against the defective gene is enhanced.

Preferred diseases or disorders which also can be treated according to the methodologies of the present invention are those for which a specific protein or proteins have been identified as playing a role (e.g., a causative role) in said disease or disorder. Normally expressed or functioning proteins as well as aberrantly expressed or functioning proteins playing a role in the disease or disorder make desirable targets for gene-specific targeting as described herein. For example, proteins playing a role in cellular functions such as cellular differentiation, inter- or intra-cellular communication; tissue function, such as cardiac function or musculoskeletal function; systemic responses in an organism, such as nervous system responses, hormonal responses (e.g., insulin response), or immune responses; and protection of cells from toxic compounds (e.g., carcinogens, toxins, or mutagens) make desirable targets for gene-specific targeting as described herein. Exemplary diseases or disorders targeted by the methodologies of the present invention include, but are not limited to CNS disorders such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, seizure disorders, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Further exemplary disorders include cardiac-related disorders or cardiovascular system disorders including, but not limited to, arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, and arrhythmia.

Further exemplary disorders include disorders of the musculoskeletal system such as paralysis and muscle weakness, e.g., ataxia, myotonia, and myokymia.

Further exemplary disorders include cellular apoptosis disorders. Cellular apoptosis disorders include those disorders that affect cell apoptosis processes. As used herein, a “cellular apoptosis process” is a process by which a cell undergoes programmed cell death.

Further exemplary disorders include hormonal disorders, such as conditions or diseases in which the production and/or regulation of hormones in an organism is aberrant. Examples of such disorders and diseases include type I and type II diabetes mellitus, pituitary disorders (e.g., growth disorders), thyroid disorders (e.g., hypothyroidism or hyperthyroidism), and reproductive or fertility disorders (e.g., disorders which affect the organs of the reproductive system, e.g., the prostate gland, the uterus, or the vagina; disorders which involve an imbalance in the levels of a reproductive hormone in a subject; disorders affecting the ability of a subject to reproduce; and disorders affecting secondary sex characteristic development, e.g., adrenal hyperplasia).

Further exemplary disorders include immune disorders, such as autoimmune disorders or immune deficiency disorders, e.g., congenital X-linked infantile hypogammaglobulinemia, transient hypogammaglobulinemia, common variable immunodeficiency, selective IgA deficiency, chronic mucocutaneous candidiasis, or severe combined immunodeficiency.

Further exemplary disorders include disorders affecting any tissue(s) in which a target protein is expressed.

These methods can be used alone, or in combination with other treatments. Further, the other treatments can be started prior to, concurrent with, or after the administration of the compositions of the instant invention. Accordingly, the methods of the invention can further include the step of administering a second treatment, such as for example, a second treatment for the disease or disorder or to ameliorate side effects of other treatments. Such second treatment can include, e.g., radiation, chemotherapy, transfusion, operations (e.g., excision to remove tumors), and gene therapy. Additionally or alternatively, further treatment can include administration of drugs to further treat the disease (e.g., HIV drug cocktails), or to treat a side effect of the disease or other treatments (e.g., anti-nausea drugs).

With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market.

More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

The language “therapeutically effective amount” is that amount necessary or sufficient to produce the desired physiologic response. The effective amount may vary depending on such factors as the size and weight of the subject, or the particular compound. The effective amount may be determined through consideration of the toxicity and therapeutic efficacy of the compounds by standard pharmaceutical procedures in cell cultures or experimental animals, e.g., for determining the LD₅₀ (The Dose Lethal To 50% Of The Population) and the ED₅₀ (the dose therapeutically effective in 50% of the population). The dose ratio between toxic and therapeutic effects is the therapeutic index and it may be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to unaffected cells and, thereby, reduce side effects.

1. Prophylactic Methods

In one aspect, the invention provides a method for preventing in a subject, a disease or condition associated with an aberrant or unwanted target gene expression or activity, by administering to the subject a therapeutic agent (i.e., a RNAi modulating compound of the invention). Subjects at risk for a disease which is caused or contributed to by aberrant or unwanted target gene expression or activity can be identified by, for example, any or a combination of diagnostic or prognostic assays as described herein. Administration of a prophylactic agent can occur prior to the manifestation of symptoms characteristic of the target gene aberrancy, such that a disease or disorder is prevented or, alternatively, delayed in its progression.

2. Therapeutic Methods

Another aspect of the invention pertains to methods of modulating target gene expression, protein expression or activity for therapeutic purposes. Accordingly, in an exemplary embodiment, the modulatory method of the invention involves contacting a cell capable of expressing target gene with a therapeutic agent (e.g., an RNAi modulating compound of the invention, and optionally an RNAi agent). These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a target gene polypeptide or nucleic acid molecule. Inhibition of target gene activity is desirable in situations in which target gene is abnormally unregulated and/or in which decreased target gene activity is likely to have a beneficial effect.

The present invention provides a method for treating a subject that would benefit from administration of a compound of the present invention. Any therapeutic indication that would benefit from RNAi modulation can be treated by the methods of the invention. The present invention provides methods of treating a subject at risk for or having a disease or disorder associated with or related to normal or aberrant expression of a gene. The method includes the step of administering to the subject a compound or compositions of the invention, such that RNAi against the gene is modulated. The disease or disorder can be any of the diseases or disorders discussed herein.

The composition administered can include one or more RNAi agent (e.g., siRNA and an expression vector encoding one or more siRNA), and optionally, other machinery needed to form or stabilize a RNA-induced silencing complex (RISC). Another example is use of the invention to treat influenza by activating RNAi against viral or cellular genes that facilitate infection.

The methods of the invention can also be used to treat dominant genetic diseases caused by aberrant expression of proteins and/or any of the other diseases discussed above.

The compounds and compositions of the invention can be administered to a subject alone or in combination with a second therapy, e.g., chemotherapy or radiation therapy. The compounds and compositions of the invention can be administered to a subject prior to, at the same time, or after a second therapy is administered.

The invention provides methods of modulating RNAi that feature contacting a cell with a compound or composition of the invention, with some preferred molecules having the structures set forth in FIGS. 1A-D herein, such that modulation of RNAi is achieved. The compound can be an RNAi activating compound where repression of the target gene is desired, or the compound can be an RNAi inhibiting compound where repression of RNAi is desired. The phrase “contacting a cell” includes contacting a cell either in vitro or in vivo. Contacting cells in vivo includes administering a compound (or composition comprising said compound) to a subject such that said compound in such a manner that the compound comes into proximity with the intended target cells, allowing the compound to perform its intended function.

The present invention also features therapeutic methods of modulating RNAi that feature administering to a subject in need thereof, an RNAi modulation compound of the present invention, with preferred molecules having the structures set forth in the FIG. 1A-D herein, said molecule having the property of modulating RNAi.

3. Pharmacogenomics

The therapeutic agents of the invention can be administered to individuals to treat (prophylactically or therapeutically) disorders associated with aberrant or unwanted target gene activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a therapeutic agent as well as tailoring the dosage and/or therapeutic regimen of treatment with a therapeutic agent.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect.

Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach,” can be utilized to identify genes that predict drug response. According to this method, if a gene that encodes a drugs target is known (e.g., a target gene polypeptide of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a therapeutic agent of the present invention can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a therapeutic agent, as described herein.

Therapeutic agents can be tested in an appropriate animal model. For example, a RNAi modulating compound can be used in an animal model to determine the efficacy, toxicity, or side effects of treatment with said agent. Alternatively, a therapeutic agent can be used in an animal model to determine the mechanism of action of such an agent. For example, an agent can be used in an animal model to determine the efficacy, toxicity, or side effects of treatment with such an agent. Alternatively, an agent can be used in an animal model to determine the mechanism of action of such an agent.

4. Gene Therapy

In yet another aspect, the present invention provides a method of treating a subject at risk for or having a disease or disorder associated with normal or aberrant expression of a gene and/or expression of a defective gene. The method can include the step of administering a compound of the present invention to the subject such that RNAi against the gene is modulated, (e.g., wherein RNAi is enhanced against aberrant expression of a gene or expression of a defective gene). The method can also include the step of administering the compound in the presence of an expression vector that, e.g., expresses a non-defective gene to replace a defective gene.

Nucleic acid molecules, for example, nucleic acid molecules encoding (i.e., for the generation of) RNAi agents or target RNAs or proteins, can be introduced into cells or into organism using a variety of art-recognized procedures. The nucleic acid molecules being introduced are often in the form of a plasmid or vector. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Alternatively, linear nucleic acid molecules, (e.g., DNA constructs or cassettes) can be introduced. In certain embodiments, the nucleic acid molecules comprise a transgene designed for stably integration into the genome of a host cell (e.g., a host cell within an organism).

The vectors of the invention generally comprise nucleic acid molecule encoding, for example, an RNAi agent or target mRNA or protein, operatively linked to one or more regulatory sequences (e.g., promoter sequences). The phrase “operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters (e.g., RNA polymerase sensitive promoters), enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). Other elements included in the design of a particular expression vector can depend on such factors as the choice of the host cell to be transformed, the level of expression of protein desired, etc. The expression vectors of the invention can be introduced into host cells to thereby produce nucleic acids, proteins or peptides, including fusion proteins or peptides, encoded by nucleic acids as described herein.

The vectors described herein can be introduced into cells or tissues by any one of a variety of known methods within the art. Such methods are described for example in Sam brook et al., Molecular Cloning: A Laboratory Manual, Cold Spring Harbor Laboratory, New York (1992), which is hereby incorporated by reference. See, also, Ausubel et al., Current Protocols in Molecular Biology, John Wiley and Sons, Baltimore, Md.(1989). The methods include, for example, stable or transient transfection, lipofection, electroporation and infection with recombinant viral vectors.

In preferred embodiments, retrovirus vectors and/or adeno-associated virus vectors are used as a recombinant gene delivery system for the transfer of exogenous genes in vivo or in vitro. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller (1990) Blood 76:271). A replication defective retrovirus can be packaged into virions which can be used to infect a host or target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) 20 Greene Publishing Associates (1989) Sections 9.10-9.14 and other standard laboratory manuals. See also Hitt et al, “Construction and propagation of human adenovirus vectors,” in Cell Biology: A Laboratory Handbook, Ed. J. E. Celis., Academic Press. 2.sup.nd Edition, Volume 1, pp: 500-512, 1998; Hitt et al, “Techniques for human adenovirus vector construction and characterization,” in Methods in Molecular Genetics, Ed. K. W. Adolph, Academic Press, Orlando, Fla., Volume 7B, pp:12-30, 1995; Hitt, et al., “Construction and propagation of human adenovirus vectors,” in Cell Biology: A Laboratory Handbook,” Ed. J. E. Celis. Academic Press. pp:479-490, 1994, also hereby incorporated by reference.

Examples of suitable retroviruses include pLJ, pZIP, pWE and pEM which are known to those the skilled artisan. Examples of suitable packaging virus lines for preparing both ecotropic and amphotropic retroviral systems include Psi-Crip, Psi-Cre, Psi-2 and Psi-Am. Retroviruses have been used to introduce a variety of genes into many different cell types, including epithelial cells, in vitro and/or in vivo (see for example Eglitis et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad Sci. USA 87:614130 6145; Cuber et al. (1991) Proc. Natl. Acad. Sci. USA 88:8039-8043; Ferry et al. (1991) Proc. Natl. Acad Sci. USA 88:8377-8381; Chowdhury et al. (1991) Science 254:1802-1805; van Beusechem et al. (1992) Proc. Natl. Acad Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

In another example, recombinant retroviral vectors capable of transducing and expressing genes inserted into the genome of a cell can be produced by transfecting the recombinant retroviral genome into suitable packaging cell lines such as PA317 and Psi-GRIP (Cornette et al. (1991) Human Gene Therapy 2:5-10; Cone et al. (1984) Proc. Natl. Acad. Sci USA 81:6349). Recombinant adenoviral vectors can be used to infect a wide variety of cells and tissues in susceptible hosts (e.g., rat, hamster, dog, and chimpanzee) (Hsu et al. (1992) J. Infectious Disease 166:769), and also have the advantage of not requiring mitotically active cells for infection.

Another viral gene delivery system useful in the present invention also utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616), Rosenfeld et al. (1991) Science 252:431-434 and Rosenfeld et al. (1992) Cell 68:143-155). Suitable adenoviral vectors derived from so the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) supra). Furthermore, the virus particle is relatively stable and amenable to purification and concentration, and as above, can be modified to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267).

Yet another viral vector system useful for delivery of foreign nucleic acid molecules (e.g., transgenes) is the adeno-associated virus (AAV). Adeno-associated virus is a naturally occurring defective virus that requires another virus, such as an adenovirus or a herpes virus, as a helper virus for efficient replication and a productive life cycle. For a review, see Muzyczka et al. (1992, Curr. Topics in Micro. and Imunol. 158:97-129). It is also one of the few viruses that may integrate its DNA into non-dividing cells, and exhibits a high frequency of stable integration (see for example Flotte et al. (1992, Am. J. Respir. Cell. Mol. Biol. 7:349-356; Samulski et al. , 1989, J. Virol. 63:3822-3828; and McLaughlin et al. (1989, J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad Sci. USA 81:6466 6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to nucleic acid molecules of the invention (e.g., nucleic acid molecules encoding RNAi agents) in the tissue of an animal. Most non-viral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject gene of the invention by the targeted cell. Exemplary gene delivery systems of this type include liposomal derived systems, polylysine conjugates, and artificial viral envelopes. Other embodiments include plasmid injection systems such as are described in Meuli et al. (2001) J. Invest. Dermatol. 116(1):131-135; Cohen et al. (2000) Gene Ther. 7(22):1896-905; and Tam et al. (2000) Gene Ther. 7(21):1867-74.

In a representative embodiment, a nucleic acid molecule of the invention (e.g., a gene encoding RNAi agent of the invention) can be entrapped in liposomes bearing positive charges on their surface (e.g., lipofectins) and (optionally) which are tagged with antibodies against cell surface antigens of the target tissue (Mizuno et al. (1992) No Shinkei Geka, 20:547-551; PCT publication WO91/06309; Japanese patent application 1047381; and European patent publication EP-A-43075).

Another aspect of the invention pertains to host cells into which a host construct of the invention has been introduced, i.e., a “recombinant host cell.” It is understood that the term “recombinant host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell, although eukaryotic cells are preferred. Exemplary eukaryotic cells include mammalian cells (such as Chinese hamster ovary cells (CHO) or COS cells). Other suitable host cells are known to those skilled in the art.

The host cells of the invention can also be used to produce nonhuman transgenic animals. The nonhuman transgenic animals can be used in screening assays designed to identify agents or compounds, e.g., drugs, pharmaceuticals, etc., which are capable of ameliorating detrimental symptoms of selected disorders, such as disease and disorders associated with mutant or aberrant gene expression, gain-of-function mutants and neurological diseases and disorders.

The present invention is also not limited to the use of the cell types and cell lines used herein. Cells from different tissues or different species (human, mouse, etc.) are also useful in the present invention.

### VI. Pharmaceutical Compositions

The invention pertains to uses of the above-described agents for therapeutic treatments as described infra. Accordingly, the compounds of the present invention can be incorporated into pharmaceutical compositions suitable for administration. Such compositions typically comprise the compounds of the invention and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The compounds of the present invention may further contain a basic functional group, such as amino or alkylamino, and are, thus, capable of forming pharmaceutically acceptable salts with pharmaceutically acceptable acids. The term “pharmaceutically acceptable salt” in this respect, refers to the relatively non-toxic, inorganic and organic acid addition salts of compounds of the present invention. These salts may be prepared in situ during the final isolation and purification of the compounds of the invention, or by separately reacting a purified compound of the invention in its free base form with a suitable organic or inorganic acid, and isolating the salt thus formed.

Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (see, e.g., Berge et al. (1977) “Pharmaceutical Salts”, J. Pharm. Sci. 66:1-19).

In other cases, the compounds of the present invention may contain one or more acidic functional groups and, thus, are capable of forming pharmaceutically acceptable salts with pharmaceutically acceptable bases. The term “pharmaceutically acceptable salt” in these instances refers to the relatively non-toxic, inorganic and organic base addition salts of compounds of the present invention. These salts may likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts, and the like.

Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants may also be present in the compositions.

Examples of pharmaceutically acceptable antioxidants, which may also be present in formulations of therapeutic compounds of the invention, include water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal or parenteral administration. The formulations may conveniently be presented in unit dosage form and may be prepared by any methods well known in the art of pharmacy. The amount of active ingredient which may be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) or as mouth washes and the like, each containing a predetermined amount of a compound of the present invention as an active ingredient. A compound of the present invention may also be administered as a bolus, electuary, or paste.

In solid dosage forms of the invention for oral administration (capsules, tablets, pills, dragees, powders, granules, and the like), the active ingredient is mixed with one or more pharmaceutically acceptable carriers, such as sodium citrate or dicalcium phosphate, or any of the following: fillers or extenders, such as starches, lactose, sucrose, glucose, mannitol, or silicic acid; binders, such as, for example, carboxymethylcellulose, alginates, gelatin, polyvinyl pyrrolidone, sucrose or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents.

In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes or microspheres.

They may be sterilized by, for example, filtration through a bacteria-retaining filter, or by incorporating sterilizing agents in the form of sterile solid compositions which may be dissolved in sterile water, or some other sterile injectable medium immediately before use.

These compositions may also optionally contain opacifying agents and may be of a composition that they release the active ingredient(s) only, or preferentially, in a certain portion of the gastrointestinal tract, optionally, in a delayed manner. Examples of embedding compositions which may be used include polymeric substances and waxes. The active ingredient may also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert dilutents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert dilutents, the oral compositions may also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a compound of this invention, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms may be made by dissolving or dispersing the compound in the proper medium. Absorption enhancers may also be used to increase the flux of the compound across the skin. The rate of such flux may be controlled by either providing a rate controlling membrane or dispersing the active compound in a polymer matrix or gel.

Ophthalmic formulations, eye ointments, powders, solutions and the like, are also within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release may be controlled. Examples of other biodegradable polymers include poly(orthoesters) and poly(anhydrides). Depot injectable formulations are also prepared by entrapping the drug in liposomes or microemulsions which are compatible with body tissue.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

The pharmaceutical compositions can be included in a container along with one or more additional compounds or compositions and instructions for use. For example, the invention also provides for packaged pharmaceutical products containing two agents, each of which exerts a therapeutic effect when administered to a subject in need thereof. The first agent is a composition comprising one or more RNAi modulating compounds. The second agent can be an RNAi agent (e.g., siRNA, siRNA expression plasmid, or siRNA complex) that is useful for treating or preventing the disorder by RNAi. A pharmaceutical composition may also comprise a third agent, or even more agents yet, wherein the third (and fourth, etc.) agent can be another agent against the disorder, such as a cancer treatment (e.g., an anticancer drug and/or chemotherapy) or an HIV cocktail. In some cases, the individual agents may be packaged in separate containers for sale or delivery to the consumer. The agents of the invention may be supplied in a solution with an appropriate solvent or in a solvent-free form (e.g., lyophilized). Additional components may include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

The present invention also includes packaged pharmaceutical products containing a first agent in combination with (e.g., intermixed with) a second agent. The invention also includes a pharmaceutical product comprising a first agent packaged with instructions for using the first agent in the presence of a second agent or instructions for use of the first agent in a method of the invention. The invention also includes a pharmaceutical product comprising a second or additional agents packaged with instructions for using the second or additional agents in the presence of a first agent or instructions for use of the second or additional agents in a method of the invention. Alternatively, the packaged pharmaceutical product may contain at least one of the agents and the product may be promoted for use with a second agent.

### VII. Knockout and/or Knockdown Cells or Organisms

A further preferred use for the compounds of the present invention is a functional analysis to be carried out in eukaryotic cells, or eukaryotic non-human organisms, preferably mammalian cells or organisms and most preferably human cells. By administering suitable compounds, and optionally an RNAi agent (e.g., an siRNA and an siRNA expression vector) comprising a sequence having sufficient complementarity to a target mRNA sequence to direct target-specific RNA interference, a specific knockout or knockdown phenotype can be obtained in a target cell, e.g., a cancerous cell.

In one embodiment, a selected gene can be knocked down by RNAi that is enhanced by the addition of an RNAi activator compound and the resultant phenotype can be observed. The amount of RNAi activating compound can be chosen to enhance RNAi to a desired level. For example, an amount of RNAi activating compound useful in enhancing RNAi activity by 10%, 20%, 30%, etc. can be employed. Such amounts can be determined by incubating varying amounts of compound introduced to a cell or organism capable of RNAi activity and measuring the change in RNAi activity as compared to a suitable control.

In another embodiment, an RNAi inhibitor can be used where knockdown of an essential gene can be lethal or toxic and may affect many pathways in the cell. Thus, in the case of a siRNA that is provided to a cell and exhibits undesirable toxic effects, such effects can be modulated by contacting the cell with a compound of the invention. Suitable concentrations of compound for this purpose include concentrations that do not maximally inhibit RNAi activity. An amount of a compound that ameliorates the effect of RNAi activity can be determined by incubating an amount of compound that inhibits RNAi by less than 100%. For example, a RNAi inhibitor compound that is useful for reducing the RNAi effect of a siRNA can inhibit RNAi activity by less than, e.g., 90%, 75%, 50%, 25%, or 10%.

Thus, a further subject matter of the invention is a eukaryotic cell or a eukaryotic non-human organism exhibiting a target gene-specific knockout or knockdown phenotype comprising a fully or at least partially deficient expression of at least one endogenous target gene that includes a RNAi modulating compound or composition of the present invention. The cell or organism can include exogenous or endogenous siRNA comprising a sequence having sufficient complementarity to a target gene to mediate target-specific RNAi. Additionally or alternatively, the cell or organism can be transfected with at least one vector comprising DNA encoding a siRNA molecule capable of inhibiting the expression of the target gene. It should be noted that the present invention allows a target-specific knockout or knockdown of several different endogenous genes due to the specificity of the siRNA.

Gene-specific knockout or knockdown phenotypes of cells or non-human organisms, particularly of human cells or non-human mammals may be used in analytical procedures, e.g., in the functional and/or phenotypical analysis of complex physiological processes such as analysis of gene expression profiles and/or proteomes. Preferably the analysis is carried out by high throughput methods using oligonucleotide based chips.

Using RNAi based knockout or knockdown technologies, the expression of an endogenous target gene may be inhibited in a target cell or a target organism. The endogenous gene may be complemented by an exogenous target nucleic acid coding for the target protein or a variant or mutated form of the target protein, e.g., a gene or a DNA, which may optionally be fused to a further nucleic acid sequence encoding a detectable peptide or polypeptide, e.g., an affinity tag, particularly a multiple affinity tag.

### VIII. Functional Genomics and/or Proteomics

Further applications for the compounds and compositions of the present invention include the analysis of gene expression profiles and/or proteomes. In one embodiment, an analysis of a variant or mutant form of one or several target proteins is carried out, wherein said variant or mutant forms are reintroduced into the cell or organism by an exogenous target nucleic acid. The combination of knockout of an endogenous gene and rescue by using mutated, e.g., partially deleted exogenous target has advantages compared to the use of a knockout cell. Further, this method is particularly suitable for identifying functional domains of the targeted protein. In a further preferred embodiment a comparison, e.g., of gene expression profiles and/or proteomes and/or phenotypic characteristics of at least two cells or organisms is carried out. These organisms are selected from: (i) a control cell or control organism without target gene inhibition, (ii) a cell or organism with target gene inhibition and (iii) a cell or organism with target gene inhibition plus target gene complementation by an exogenous target nucleic acid.

Another utility of the present invention could be a method of identifying gene function in an organism comprising the use of the RNAi modulating compounds to enhance or inhibit the activity of a target gene of previously unknown function. Instead of the time consuming and laborious isolation of mutants by traditional genetic screening, functional genomics would envision determining the function of uncharacterized genes by employing the invention to reduce the amount and/or alter the timing of target gene activity.

Accordingly, the present invention provides a method for analyzing the gene expression profile in a cell or an organism. The method includes the step of contacting the cell or the organism with a compound or composition or the invention, wherein the compound or composition modulates RNAi against at least one target gene, and analyzing the affect of the RNAi modulation against the target gene on the gene expression in the cell or organism.

The invention could be used in determining potential targets for pharmaceutics, understanding normal and pathological events associated with development, determining signaling pathways responsible for postnatal development/aging, and the like. The increasing speed of acquiring nucleotide sequence information from genomic and expressed gene sources, including total sequences for the yeast, D. melanogaster, and C. elegans genomes, can be coupled with the invention to determine gene function in an organism (e.g., nematode). The preference of different organisms to use particular codons, searching sequence databases for related gene products, correlating the linkage map of genetic traits with the physical map from which the nucleotide sequences are derived, and artificial intelligence methods may be used to define putative open reading frames from the nucleotide sequences acquired in such sequencing projects. A simple assay would be to inhibit gene expression according to the partial sequence available from an expressed sequence tag (EST). Functional alterations in growth, development, metabolism, disease resistance, or other biological processes would be indicative of the normal role of the EST's gene product.

The ease with which small molecules can be introduced into an intact cell/organism containing the target gene allows the present invention to be used in high throughput screening (HTS). Solutions containing RNAi modulating compounds, and optionally siRNAs (and/or siRNA expression vectors) that are capable of inhibiting the different expressed genes, can be placed into individual wells positioned on a microtiter plate as an ordered array, and intact cells/organisms in each well can be assayed for any changes or modifications in behavior or development due to inhibition of target gene activity.

The function of the target gene can be assayed from the effects it has on the cell/organism when gene activity is inhibited. This screening could be amenable to small subjects that can be processed in large number, for example: arabidopsis, bacteria, drosophila, fungi, nematodes, viruses, zebrafish, and tissue culture cells derived from mammals. A nematode or other organism that produces a colorimetric, fluorogenic, or luminescent signal in response to a regulated promoter (e.g., transfected with a reporter gene construct) can be assayed in an HTS format.

The present invention may be useful in allowing the inhibition of essential genes. Such genes may be required for cell or organism viability at only particular stages of development or cellular compartments. The functional equivalent of conditional mutations may be produced by inhibiting activity of the target gene when or where it is not required for viability. The invention allows addition of the RNAi modulating compounds of the invention at specific times of development and locations in the organism without introducing permanent mutations into the target genome.

The present invention also provides a method of analyzing the RNAi activation pathway in a cell or organism. The method includes contacting the cell or the organism with a compound or composition of the invention; and analyzing the cell or organism for the affect of the compound or composition on a RNAi-mediated pathway. The compound can be ligated to an extraction media in order to extract compounds bound to or associated with the compound. Extractions can be made at various times and locations in the cell or organism and analyzed to study the RNAi-mediated pathway.

In yet another embodiment, a phenotype that is observed due to the effect of RNAi knockdown of a specific gene (i.e., a target gene that is inhibited by a targeted siRNA) can be reversed by inhibiting the RNAi effect. Such a result would confirm the function of the targeted gene. In such experiments, an RNAi agnet (e.g., a siRNA) that is targeted to a specific gene is introduced into a cell. Such procedures are known in the art. For example, see Elbashir et al. (2001, Nature, 411:494-498). To confirm that the effect of a targeted RNAi agent on a cell (e.g., on expression of the target gene's protein product) is due to RNAi, a compound of the invention is added to the medium in which the cell is cultured. The compound can be added either before or after introducing an RNAi agent into the cell. In general, the ATPA is added within 6 hours after transfection. The concentration of the compound in the medium is generally between about 1 μM-100 μM, e.g., 10 μM-50 μM. After a suitable incubation time, i.e., sufficient time for the targeted siRNA to inhibit expression of the targeted gene, the expression of the targeted gene in a control culture, in a culture that received targeted RNAi agent, and in a culture that received targeted RNAi agent and the compound can be compared. In this case, a control culture is generally a culture that that did not receive RNAi agent, or RNAi agent+compound, and/or a culture that received an RNAi agent that is not targeted to an expressed gene. Inhibition of expression (detected either by detection of RNA expression, protein expression, activity of the protein, or a physiologic effect of the protein) of a target gene in the presence of the targeted RNAi agent that is reversed in the presence of targeted RNAi agent and the compound indicates an effect resulting from RNAi. Thus, the effect is specific to expression of the targeted gene.

### IX. Screening Assays for Pharmacological Agents

The methods of the invention are also suitable for use in methods to identify and/or characterize potential pharmacological agents, e.g., identifying new pharmacological agents from a collection of test substances and/or characterizing mechanisms of action and/or side effects of known pharmacological agents.

Thus, the present invention also relates to a system for identifying and/or characterizing pharmacological agents. For example, the method can be used to screen for a pharmacological agent for treating a disease or disorder associated with the normal or aberrant expression of a gene. The system generally includes: (a) a eukaryotic cell or a eukaryotic non-human organism capable of expressing at least one endogenous or exogenous (e.g., viral) target gene coding for said target protein, (b) at least one RNAi agent capable of inhibiting the expression of said at least one endogenous target gene, (c) an RNAi modulator of the present invention, and (d) a test substance or a collection of test substances wherein pharmacological properties of said test substance or said collection are to be identified and/or characterized. Such an assay can also be used to identify or characterize pharmacological agents that attenuate the affect of the RNAi activity. For example, the system can be designed to simulate a condition of a disease or disorder, e.g., reduced expression of a gene, and the test substances can be characterized based on their ability to treat that disease or disorder. The test substance can also be used to identify or characterized super inhibitors (or activators) that modulate RNAi even in the presence of a known RNAi activator (or inhibitor). The system also can be used to identify or characterized modulators that can be used with known modulators to identify or characterize, e.g., a symbiotic effect between two modulators.

The compositions of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner USP 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.)).

### X. Method of Affinity Purification of RNAi Components

The compounds of the invention can also be used to affinity purify proteins involved in the RNAi mechanism. This includes affinity purification of such proteins from various organisms, e.g., worms (such as Caenorhabditis elegans), insects (such as Drosophila melanogaster), and mammals (e.g., mice, rats, domestic animals, and humans). Such molecules provide insight into the mechanism of RNAi in mammalian cells and additional targets for compounds that inhibit or activate RNAi. Methods for attaching a compound to a substrate for use in purification methods and methods for affinity purification of proteins are known in the art. Exemplary compounds include the following structures made by attaching biotin to ATPA 18 and ATPA 18K.

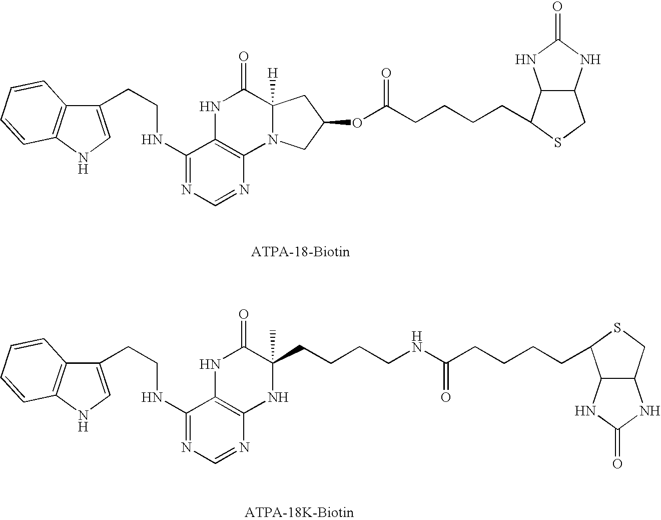

An exemplary method of using a compound of the invention for affinity purification is provided in Example 9.

### XI. Trifunctionalized Derivatives and Method of Purification of RNAi Components

The present invention also provides novel trifunctionalized compounds, which can be used, e.g., in cross-linking experiments, to affinity-purify RNAi components that do not stably bind to a RNAi modulating compound. The trifunctionalized compounds of the invention include a cross-linking moiety (e.g., a photocrosslinking group such as 9-(5-Iodo-pentyloxy)-furo[3,2-g]chromen-7-one), and an immobilization moiety (e.g., biotin).

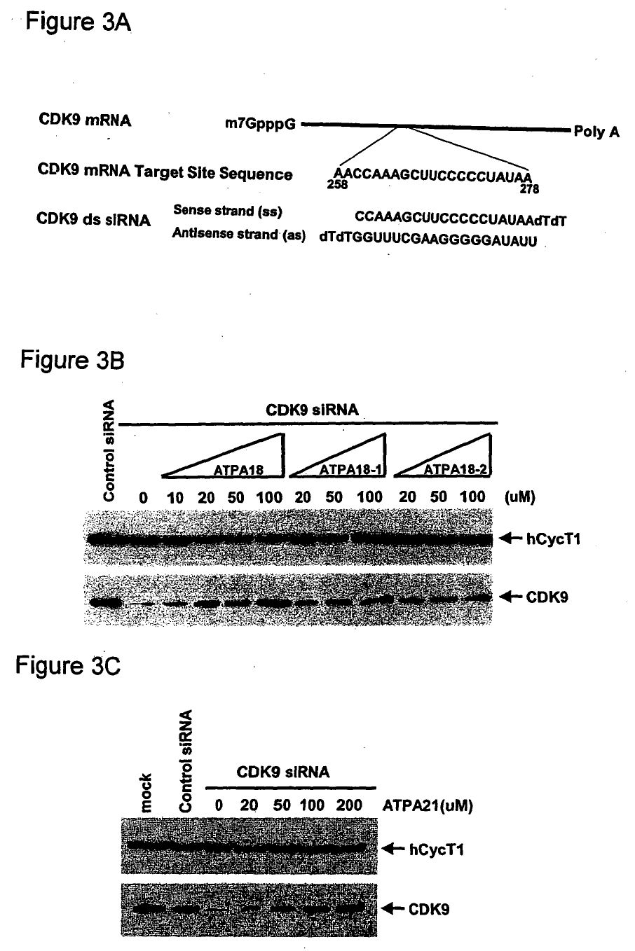

Methods for attaching the functional groups to a compound are known in the art. Exemplary methods and products of trifunctionalization are illustrated in FIGS. 3 and 4. In FIGS. 3 and 4, ATPA18K is trifunctionalized, but any other compound of the present invention also can readily be trifunctionalized using standard techniques. In addition, any other known crosslinkers can be used in the present invention, including but are not limited to, benzophenones, nitrogen mustards, and aryl-azides. Psoralens and psoralen derivatives that can be used in the present invention include, but are not limited to, 8-hydroxypsoralen, 8-((3-Idodopropyl-1)oxy)psoralen, aminomethyl psoralen, amino acid-modified psoralens, psoralen derivatives with modified stereochemistry, and psoralen derivatives with solubility in aqueous buffers.

The trifunctionalized compounds of the invention can be added to cells or cell extracts such that the compounds are brought into proximity with nucleic acid molecules, e.g., RNA or DNA molecules (e.g., siRNA and other RNAi components). The compounds are then exposed to long wave UV (360 nm) to crosslink or immobilize components that have an affinity to the ATPA. The trifunctionalized compound, crosslinked with the bound RNAi component, can readily be immobilized on avidin-coated magnetic beads and removed and analyzed. Optionally, further reactions can be carried out on beads or solution phase. Trifunctionalized compounds may be useful for isolating, e.g., target RNA crosslinked to RNAi agents, optionally associated with other RNAi components. The ATPA moiety may be useful for modulating the activity of RNAi components, e.g., inhibiting the activity or otherwise affecting RNAi components (e.g., affecting the proximity of the components, e.g., with the RNAi agents).

### EXEMPLIFICATION

This invention is further illustrated by the following examples which should not be construed as limiting.

### MATERIALS AND METHODS

siRNA Preparation

21 -nucleotide siRNA were chemically synthesized as 2′ bis (acetoxyethoxy)-methyl ether-protected oligos by Dharmacon (Lafayette, Colo.). Synthetic oligonucleotides were deprotected, annealed, purified, and successful duplex formation was confirmed by 20% non-denaturing polyacrylamide gel electrophoresis. All siRNAs were stored in DEPC (0.1% diethyl pyrocarbonate)-treated water at −80 ° C. siRNA duplexes were synthesized for target sequences 238-258 of EGFP mRNA and positions 277-297 of RFP mRNA relative to the start codon (FIG. 2A). The sequences of the target-specific siRNA duplexes were designed according to the manufacturer's recommendation and subjected to a BLAST search against the human genome sequence to ensure that no endogenous genes of the genome were targeted.

For RNA interference targeting to an endogenous gene, the sequence of CDK9-specific siRNA duplexes was designed using the manufacturer's recommendations (Dharmacon) and subjected to a BLAST search against the human genome sequence to ensure that only the CDK9 gene was targeted. The siRNA sequence targeting CDK9 was from position 258-278 relative to the start codon (FIG. 3A).

Culture and Transfection of Cells

HeLa cells were maintained at 37° C. in Dulbecco's modified Eagle's medium (DMEM, Invitrogen) supplemented with 10% fetal bovine serum (FBS), 100 units/ml penicillin, and 100 μg/ml streptomycin (Invitrogen). Cells were regularly passaged at subconfluence and plated 16 h before transfection at 70% confluency. Lipofectamine (Invitrogen)-mediated transient co-transfections of reporter plasmids and siRNAs were performed in duplicate 6-well plates. A transfection mixture containing 0.16 μg pEGFP-C1 and 0.33 μg pDsRed2-N1 reporter plasmids (Clontech), siRNA (as indicated in each experiment), and 10 μl lipofectamine in 1 ml serum-reduced OPTI-MEM (Invitrogen) was added to each well. To test modulator effect on RNAi, candidate compounds were added into the transfection mixture at the beginning of transfection. Cells were incubated in the transfection mixture for 6 h and further cultured in antibiotic-free DMEM containing inhibitors at indicated concentrations. Cells were treated under the same conditions without siRNA or inhibitors for mock experiments. At 42 h post transfection, the transfected cells were washed twice with phosphate-buffered saline (PBS, Invitrogen), flash frozen in liquid nitrogen, and stored at −80° C. for reporter gene assays.

Dual Fluorescence Reporter Gene Assays

pEGFP-C1, pDsRed1-N1 reporter plasmids and 50 nM siRNA were cotransfected into HeLa cells. EGFP-C1 encoded enhanced green fluorescence protein (GFP), while DsRed1-N1 encoded red fluorescence protein (RFP). Cells were harvested as described above and lysed in ice-cold reporter lysis buffer (Promega) containing protease inhibitor (complete, EDTA-free, 1 tablet/10 ml buffer, Roche Molecular Biochemicals). After clearing the resulting lysates by centrifugation, protein in the clear lysate was quantified by Dc protein assay kit (Bio-Rad). 120 μg of total cell lysate in 160 μl reporter lysis buffer was measured by fluorescence spectrophometry (Photo Technology International). The slit widths were set at 4 nm for both excitation and emission. All experiments were carried out at room temperature. Fluorescence of GFP in cell lysates was detected by exciting at 488 nm and recording from 498-650 nm. The spectrum peak at 507 nm represents the fluorescence intensity of GFP. Fluorescence of RFP in the same cell lysates was detected by exciting at 568 nm and recording from 588 nm-650 nm; the spectrum peak at 583 nm represents the fluorescence intensity of RFP. The fluorescence intensity ratio of target (GFP) to control (RFP) fluorophore was determined in the presence of siRNA duplex.

Improved Dual Fluorescence Assay

In an improved dual fluorescence reporter assay, EGFP-C1 (encoding enhanced GFP), and DsRed2-N1 (encoding DsRed2 RFP, a DsRed variant that has been engineered for faster maturation and lower non-specific aggregation) is employed. The extinction coefficient of DsRed2 is 43,800 (M⁻¹cm⁻¹) and the quantum yield is 0.55, a significant quantitative increase when compared to the DsRed1 vector used in the dual fluorescence assay. To quantify RNAi effects, cell lysates are prepared from siRNA duplex-treated cells at 42 h post-transfection, as described previously (Y. L. Chiu, T. M. Rana, Mol Cell 10, 549-561. (2002)). 240 μg of total cell lysate in 160 μl reporter lysis buffer is measured by fluorescence spectrophotometry (Photo Technology International). The slit widths are set at 4 nm for both excitation and emission. All experiments are carried out at room temperature. GFP fluorescence in cell lysates was detected by exciting at 488 nm and recording from 498-650 nm. The spectrum peak at 507 nm represents the fluorescence intensity of GFP. RFP fluorescence in the same cell lysates is detected by exciting at 568 nm and recording from 588-650 nm. The spectrum peak at 583 nm represents the fluorescence intensity of RFP. The fluorescence intensity ratio of target (GFP) to control (RFP) fluorophore is determined in the presence of siRNA duplexes. The ratio can be normalized, e.g., to mock treated cells or cells without siRNA duplexes such as cells with an antisense strand directed to the target gene or with neither ds-siRNA or ss-siRNA.

CDK9 RNA Interference and Western Blotting

150 nM CDK9 siRNA were transfected into HeLa cells. At 42 h post transfection, clear lysate was prepared from siRNA-treated cells and quantified as described above. Proteins in 60 μg of total cell lysate were resolved by 10% SDS-PAGE, transferred onto a polyvinylidene difluoride membrane (PVDF membrane, Bio-Rad), and probed with antibodies against CDK9 (Santa Cruz). As a loading control, the same membrane was also probed with anti-hCycT1 antibody (Santa Cruz). Protein contents were visualized with BM Chemiluminescence Blotting Kit (Roche Molecular Biochemicals). The immunoblots were exposed to x-ray film (Kodak MR-1) for various times (between 30 s and 5 min).

Biotin Pull-out Assay for siRNA Isolation from Human Cells

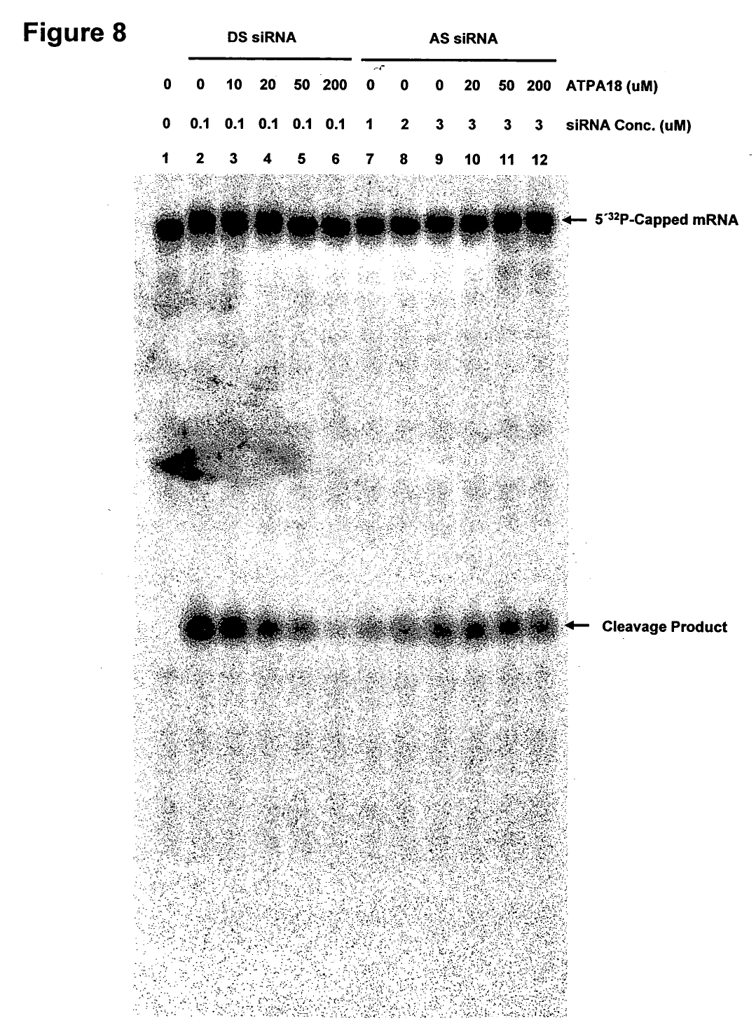

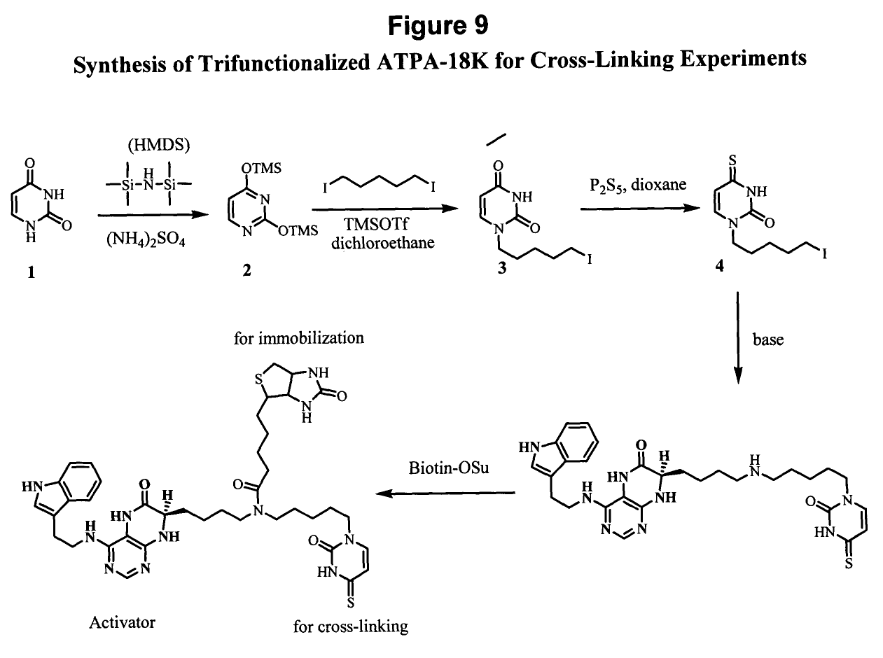

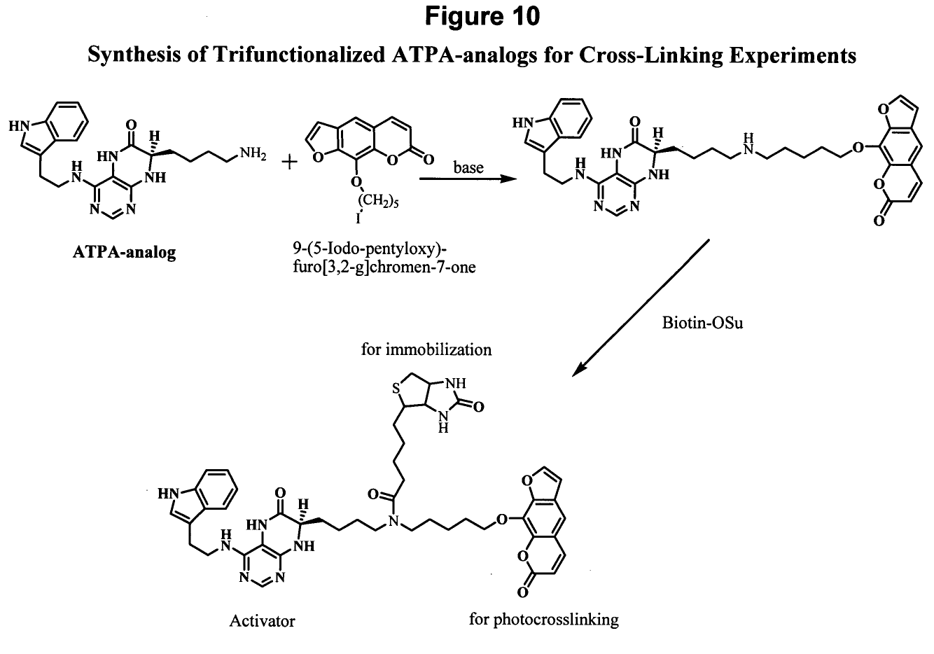

Antisense strands of the siRNA duplex were chemically synthesized and biotin-conjugated at the 3′ end (Dharmacon, Lafayette, Colo.). Synthetic oligonucleotides were deprotected and annealed with the unmodified sense strand RNA to form duplex siRNA (SS/AS3′-Biotin). HeLa cells, which had been plated at 70% confluency in 100 mm dishes, were co-transfected with duplex siRNA (˜600 pmole) and EGFP-C1 plasmid (1 μg) by a lipofectamine-mediated method with or without inhibitor treatment as described above. At 6 h post transfection, the transfected cells were washed twice with PBS (Invitrogen) and flash frozen in liquid nitrogen. The biotin pull out assay was then performed as previously described. Low molecular weight RNA was isolated from cells using a Qiagen RNA/DNA Mini Kit. Biotinylated siRNA was pulled out by incubating purified RNA with streptavidin-magnetic beads (60 μl) in TE buffer (10 mM Tris-HCl [pH 8.0], 1 mM EDTA) containing 1 M NaCl for 3 h at room temperature. The beads were washed four times with 200 μl TE buffer, resuspended in 100 μl TE buffer, and split into two equal aliquots. To one aliquot (50 μl), we added 50 units of shrimp alkaline phosphatase (SAP, Roche Molecular Biochemicals) in 1×SAP buffer and incubated the mixture at 37° C. for 1 h. The SAP reaction was then stopped by heating at 65° C. for 15 min and washed four times with 200 μl TE buffer. The other aliquot was not treated with SAP. Aliquots of beads with or without SAP treatment were incubated with 30 units T4 polynucleotide kinase (T4 PNK, Roche Molecular Biochemicals) in 30 μl 1×PNK buffer containing 0.2 mCi-³²P ATP for 1 h at 37° C. RNA products were resolved on 20% polyacrylamide-7 M urea gels and ³²P-labeled RNAs were detected by phosphorimaging (FIG. 8).

Alexa Fluor Labeling of siRNA Duplexes

Sense strand and antisense strands of the siRNAs were chemically synthesized and amino-modified at the 3′ end (Dharmacon, Lafayette, Colo.). Synthetic oligonucleotides were deprotected and purified by ethanol precipitation, adding one-tenth volume of 3M NaCl and two and half volumes of cold absolute ethanol. The oligonucleotides were dissolved in DEPC-treated H₂O at a final concentration of 4.2 mM, and then subjected to Alexa fluor labeling. Sense and antisense strands were labeled with Alexa fluor 647 and Alexa fluor 568, respectively. An oligonucleotide amine labeling kit was purchased from Molecular Probes. Labeling was performed in a 50 μl reaction containing reactive dye (pre-dissolved in 7 μl DMSO), 41 μl Labeling buffer (Molecular Probes) and 2 μl of 4.2 mM oligonucleotide stock for 6 h with gentle shaking at low speed. Alexa fluor-labeled oligonucleotides were purified by 20% polyacrylamide-7 M Urea gel electrophoresis. Duplex siRNAs were formed by annealing Alexa 568-labeled antisense strands (AS-Alexa 568) with unmodified (SS) or Alexa 647-labeled sense strands (SS-Alexa 647). Duplex siRNAs were purified and successful duplex formation was confirmed by 20% non-denaturing polyacrylamide gel electrophoresis.

Live Imaging of Cells and FRET

Duplex siRNAs SS-Alexa 647/AS-Alexa 568 were transfected into HeLa cells with or without inhibitor ATPA-18 treatment. Transfection mixtures were removed at 6 h post transfection and cells were continuously cultured in the presence or absence of ATPA-18. Cells were imaged at 8 h post transfection. Experiments were performed with a Leica confocal imaging spectrophotometer system (TCS-SP2) attached to a Leica DMIRE inverted fluorescence microscope and equipped with an argon laser (458-, 476-, 488-, 514-nm lines), two HeNe lasers (543-, 633- nm lines), an acousto-optic tunable filter (AOTF) to attenuate individual visible laser lines, and a tunable acousto-optical beam splitter (AOBS). A 63 X, 1.32 NA oil immersion objective was employed. For donor (AS-Alexa 568) detection, the fluorescence emission signal was recorded at 585±30 nm when excited at the Alexa 568 excitation wavelength (543 nm). For acceptor (SS-Alexa 647) detection, the fluorescence emission signal was recorded at 675±25 nm when excited at the Alexa 647 excitation wavelength (633 nm). For FRET signal detection, the fluorescence emission signal was recorded at 675±25 nm when excited at the Alexa 568 excitation wavelength (543 nm). To quantify FRET efficiency, photobleaching studies were performed with conventional excitation at 633 nm for Alexa 647. Photobleaching efficiency is ˜95% as determined by measuring the ratio of the Alexa 647 (acceptor) fluorescence emission signal pre- and post-photobleaching. FRET efficiency was determined by measuring the ratio of the Alexa 568 (donor) fluorescence emission signal pre- and post-photobleaching of Alexa 647 (acceptor). Image analysis was performed using Leica Confocal Software (LCS).

### EXAMPLE 1

### Synthesis of Substituted Dihydropteridinones as ATP Analogues

A library of substituted dihydropteridinones was generated utilizing the following procedure.

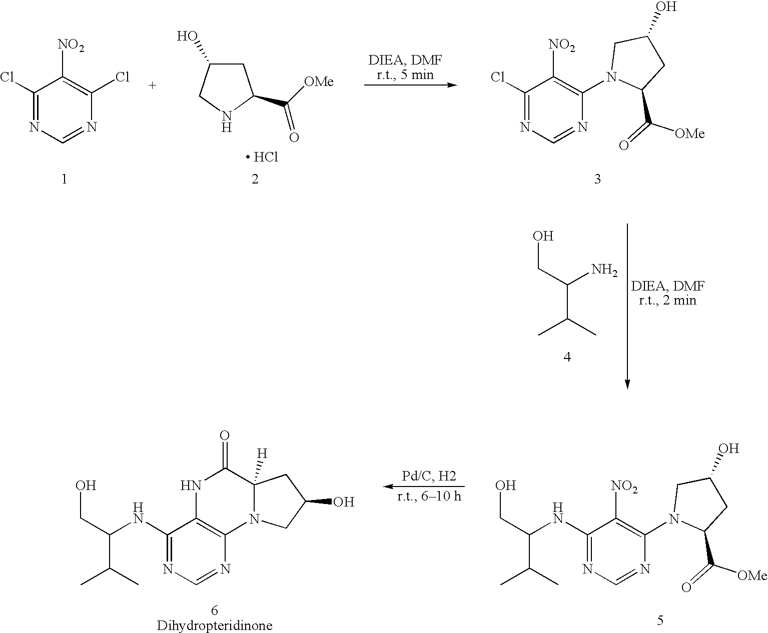

Step 1: In a 10 mL round bottomed flask, 56 μL (0.322 mmols) of diisopropylethylamine (DIEA) was added to a stirred suspension of 50mg (0.257 mmols) of 4,6-dichloro-5-nitro-pyrimidine (1) and 47 mg (0.257 mmols) of H-Hyp-OMe.HCl (2) in 0.5mL of DMF. The reaction mixture was stirred at room temperature for 5 min. during which time the reaction was completed. The solvent was then removed under reduced pressure and the crude product was used as such in the next step.

Step 2: In a 10 mL round bottomed flask, 51 mg (0.50 mmols) of L-Valinol (4) in 0.5 mL of DMF followed by 95 μL of DIEA, were added to a stirred solution 0.15 g (0.50 mmols) of product (3) in 0.5 mL of DMF. The reaction mixture was then stirred for 2 min. at room temperature and the solvent was removed under reduced pressure. The crude product was then purified on a short pad of silica gel using 60:40 ethyl acetate:hexane as eluents to obtain 89 mg (93.5% for two steps) of the pure product (5).

Step 3: In a 50 mL of round bottomed flask, 25 mg of 10% Pd/C was added, to a stirred solution of 89 mg (0.24 mmols) of the di-substituted product (5) in 15 mL of anhydrous ethanol. The reaction mixture was then stirred at room temperature under hydrogen atmosphere (balloon) for 8 h. The contents were then filtered through a short pad of celite to remove the carbon particles followed by evaporation to afford the desired product. The crude product was then purified by precipitating the pure product using 95:5 ethyl acetate:methanol to afford 65 mg (88%) of the pure product (6).

Step 4: In the case of substituents having acid labile protecting groups such as Lysine or Serine, the final product was obtained by treating the cyclized product with a mixture of 1:1 CH₂Cl₂:Trifluoroacetic acid.

The compounds that were obtained are depicted in FIGS. 1A-D.

### EXAMPLE 2

### Dual Fluorescence Reporter Gene Assay

A dual reporter system assay was developed to detect functional RNAi. For each assay, pEGFP-C1 (encoding EGFP) and pDsRed1-N1 (encoding RFP) reporter plasmid (under cytomegalovirus promoter control), and 50 nM siRNA were cotransfected into HeLa cells as described above.

The cells were harvested and lysed in ice-cold reporter lysis buffer (Promega) containing protease inhibitor (complete, EDTA-free, 1 tablet/10 mL buffer, Roche Molecular Biochemicals). The resulting lysates were cleared by centrifugation and protein amount in the clear lysate was quantified by Dc protein assay kit (Bio-Rad). 20 μg of total cell lysate in 160 μl reporter lysis buffer were subject to fluorescence measurements on a PTI (Photo Technology International) fluorescence spectrophotometer 48 hours post-transfection. The slits were set 4 nm for both excitation and emission lights. All experiments were carried out at room temperature.

Mock treatment (without siRNA) allowed efficient expression of both GFP and RFP in living HeLa cells (panels a and b of FIG. 2B). Transfection of cells with siRNA duplex targeting GFP significantly reduced GFP expression, but did not significantly reduce RFP expression (panels c and d of FIG. 2B). Transfection of cells with siRNA duplex targeting RFP significantly reduced RFP expression, and did not significantly reduce GFP expression (panels e and f of FIG. 2B).

FIG. 2C illustrates the results of quantitative analysis of the RNAi effects qualitatively shown in FIG. 2B. The fluorescence of EGFP in the cell lysate was detected by exciting at 488 nm and the emission spectrum was recorded from 498 nm to 650 nm. The spectrum peak at 507 nm represents the fluorescence intensity of EGFP. Fluorescence of RFP in the same cell lysate was detected by exciting at 568 nm and the emission spectrum was recorded from 588 nm to 650 nm. The spectrum peak at 583 nm represents the fluorescence intensity of RFP.

### EXAMPLE 3

### Identification of RNAi Activators

Candidate compounds synthesized in example 1 and depicted in FIGS. 1A-D were screened for their ability to activate RNAi as described in example 2. Briefly, cells were transfected as described above so as to express two reporter plasmids, which express two different reporter genes (a EGFP and an RFP), and were also transfected with an siRNA targeted to the GFP sequence. The cells were incubated in the presence of the RNAi activating candidate compounds. The cells were harvested at 30 hours, at which point lysates were prepared as described above. The lysates were analyzed by fluorescence spectroscopy. Fluorescence emission spectra of EGFP and RFP were detected by exciting at 488 nm and 568 nm, respectively. The fluorescence intensity ratio of target (EGFP) to control (RFP) fluorophore was determined in the presence of siRNA duplex and normalized to the ratio observed in the presence of wildtype dsRNA (control).

The results of the activation determination are qualitatively indicated in FIG. 2. Candidate compounds ATPA2, ATPA12, ATPA20 and ATPA24 activated the RNAi effect. A “*” indicates no activation, a “**” indicates activation, and a “***” indicates good activation. For the purposes of this example, a compound having less than about 7% activation was considered to have no activation.

Cell viability after treatment with the compounds of the invention was determined by the amount of the total cell lysate and was normalized to that observed in the presence of compound treatment. All the compounds tested were non-toxic.

### EXAMPLE 4

### Identification of RNAi Inhibitors

The candidate compounds (FIG. 1A-D) were screened for their ability to inhibit RNAi using methods described in example 3. The fluorescence intensity ratio of target (EGFP) to control (RFP) fluorophore was determined in the presence of siRNA duplex and normalized to the ratio observed in the presence of antisense strand as RNA (control). Normalized ratios less than 1.0 indicate specific RNAi.

FIG. 4B is a chart depicting the ratios of normalized GFP to RFP fluorescence intensity for the candidate compounds ATPA-2, ATPA-3, ATPA-6, ATPA-7, ATPA-8, ATPA-10, ATPA-11, ATPA-12, ATPA-13, ATPA-14, ATPA-16, ATPA-18, ATPA-19, ATPA-20, ATPA-21, ATPA-22, ATPA-24, antisense (as) and double-stranded (ds) RNA. The cells were treated with 25 nM duplex siRNAs and various small molecular inhibitors at 50μM in accordance with the methods described above. Structures of the two ATP analogs exhibiting inhibition effects on RNAi, ATPA-18 and ATPA-21, are shown in FIG. 4B. Antisense strand (as) for GFP had no effect on GFP expression and dsRNA exhibited interference activity. ATPA candidate compounds ATPA-18 and ATPA-21 inhibited the RNAi effect. Candidate compounds 18K and 18S (whose structures are shown in FIG. 1D) were also effective inhibitors of RNAi (results not shown).

In addition, proteins prepared from the cells were resolved on 10% SDS-PAGE. Proteins were transferred to PVDF membranes, and immunoblotted with antibodies against EGFP and DsRed1-N1. The membrane was stripped and re-probed with anti-actin antibody to check for equal loading of total proteins. These experiments demonstrate a method of screening candidate RNAi inhibitors such as candidate ATPA compounds. They also demonstrate that ATPA-18, ATPA-21, ATPA-18K, and ATPA-18S are effective inhibitors of RNAi. Furthermore, these data demonstrate the efficacy of ATPA compounds for inhibiting RNAi and the relatively low toxicity of such compounds. The modulated decrease in RNAi shown with increasing concentrations of an ATPA (see FIG. 4C), demonstrates that an ATPA can be useful for applications where it is desirable to decrease, but not entirely eliminate expression of a gene, e.g., because a complete elimination of expression caused by an siRNA is toxic to the cell. Cell viability after ATPA compound treatment was determined by the amount of the total cell lysate and was normalized to that observed in the presence of compound treatment (FIG. 4D).

### EXAMPLE 5

### CDK9 RNA Interference and Western Blotting

The ability of inhibitor compounds of the invention to affect RNAi activity and knockdown endogenous human proteins such as CDK9 was examined. CDK9 is the cyclin-dependent kinase component of the P-TEFb, CDK9-cyclinT1 complex, which is involved in regulating transcription elongation. D. H. Price, Mol Cell Biol 20, 2629-2634 (2000). The dual fluorescence assay measured the effects of the analogs on RNAi with EGFP expressed episomally. siRNAs targeting CDK9 were synthesized with 2-nt deoxythymidine overhangs at the 3′ end. These experiments were conducted similarly to those described above, except that no exogenous reporter genes were transfected into the cells. The synthesized sequence encoding a double-stranded RNA (dsRNA), targeted to CDK9 was introduced into cells. Antisense RNA was used as a control sequence.

CDK9-specific siRNA duplexes were designed as described above. FIG. 3A illustrates the position of the first nucleotide of the mRNA target site relative to the start codon of CDK9 mRNA. The sequence of the antisense strand of siRNA is exactly complementary to the mRNA target site.

200 nM CDK9 siRNA was transfected into HeLa cells. At 42 hours post transfection, clear lysate was prepared from siRNA treated cells and quantified as described above. Proteins in 60 μg of total cell lysate were resolved in 10% SDS-PAGE and transferred onto a polyvinylidene difluoride membrane (PVDF membrane, Bio-Rad) followed by immunoblotting with antibodies against CDK9 (Santa Cruz). For loading control, same membrane was also blotted with anti-hCycT1 antibody (Santa Cruz). Protein contents were visualized with BM Chemiluminescence Blotting Kit (Roche Molecular Biochemicals). The blots were exposed to x-ray film (Kodak MR-1) for various times (between 30 seconds and 5 minutes).

FIGS. 3B and 3C show that CDK9, the product of an endogenous gene, is effectively inhibited by a siRNA targeted to a CDK9 sequence and that ATPA18, ATPA18-1, ATPA18-2, and ATPA-21 are effective inhibitors of RNAi. In these experiments, CycT1 was a control showing that RNAi effect was specific for CDK9 mRNA degradation. These data show that ATPA 18 (and its derivatives) and ATPA21 are effective inhibitors of the RNAi effect to knockdown expression of an endogenous protein. In addition, these results demonstrate that ATPA-18 and ATPA-21 were effective inhibitors of RNAi at concentrations of 10-100 micromolar. These concentrations did not cause significant toxicity or cell killing.

In another series of related experiments, 150 nM CDK9 siRNA was transfected into HeLa cells with or without inhibitor treatment. At 42 h post transfection, proteins in 60 μg of total cell lysate were resolved by 10% SDS-PAGE, transferred onto polyvinylidene difluoride membrane (PVDF membrane, Bio-Rad), and probed with antibodies against CDK9 (Santa Cruz). As a loading control, the same membrane was also probed with anti-hCycT1 antibody (Santa Cruz). Protein contents were visualized with BM Chemiluminescence Blotting Kit (Roche Molecular Biochemicals). Immunoblots were exposed to x-ray film (Kodak MR-1) for various times (between 30 s and 5 min).

CDK9 double-stranded siRNA wholly complementary to CDK9 mRNA knocked down CDK9 protein levels substantially, unlike mock or mismatched CDK9 siRNA treated cells (FIG. 4C, lanes 1-2, 7-8). However, in the presence of increasing amounts of either ATPA-18 or ATPA-21 up to 100 μM, Cdk9 protein levels were restored to normal, endogenous levels in a dose-dependent manner (FIG. 4C; lanes 3-6, 10-13). These results suggest that the compounds generally interfere with RNAi regardless of whether mRNAs were expressed episomally or endogenously, and the degree of potency depended on the concentration of the ATP analog. Thus, the compounds are useful for verifying functions of endogenous genes as described supra.

### EXAMPLE 6

### Determination of Localization Patterns of siRNA

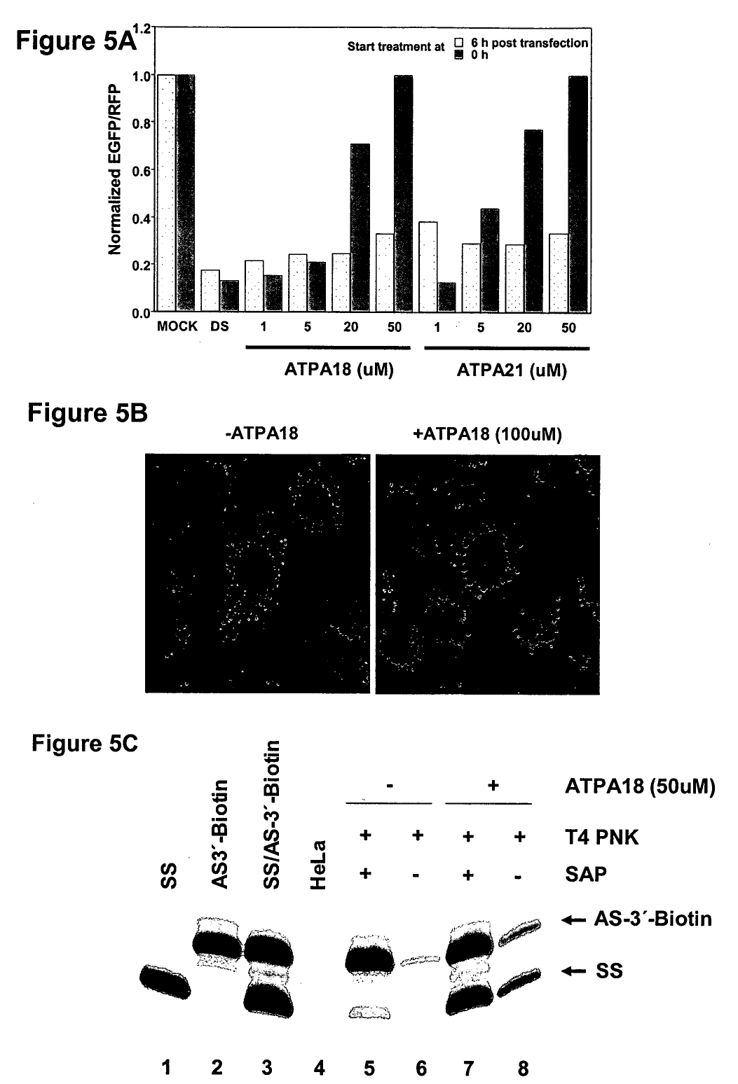

Localization patterns of siRNA were determined in the presence and absence of 100 μM ATPA-18 (FIG. 5B). FIG. 5B demonstrates that localization patterns of siRNAs were not changed by small molecular inhibitors. SS/AS-Alexa 568 duplex siRNAs with Alexa 568 labeling at the antisense strand 3′ terminus were transfected into HeLa cells and the distribution pattern was determined in the presence and absence of 100 μM ATPA-18 at 6 h post transfection. In the absence of the ATPA-18, siRNA localized to areas surrounding the nucleus and significant differences in this localization were not observed in the presence of ATPA-18 (FIG. 5B). This suggested that ATPA-18 did not disrupt the normal localization of siRNA to the cytoplasm, indicating that ATPA-18 was not inhibiting siRNA from targeting to where RNAi normally takes place in the cell.

### EXAMPLE 7

### Affects of RNAi Inhibitor on Phosphorylation State and Duplex Structure

To analyze how ATPA-18 at ATPA-21 affects the phosphorylation state and duplex structure of siRNAs, siRNA 5′ phosphorylation and the single-stranded versus double-stranded nature of siRNA treated with the compounds were analyzed. FIG. 5A is a graph demonstrating the affect of ATPA-18 and ATPA-21 on the step(s) of the RNAi pathway occurring between 0-6 h post-transfection. RNAi activity was quantified by the dual fluorescence assay and presented as the inhibition efficiency of target gene (EGFP) expression. Cells were treated with 25 nM duplex siRNAs and small molecular inhibitors were added at the beginning of transfection (0 h, dark shading) or at 6 h post transfection (light shading).

A biotin pull-out assay was run indicating that helicase activity, and not kinase activity, was the cellular target for small molecular inhibitors of RNAi in human cells. siRNA antisense strands were biotinylated on their 3′ end (designated AS-3′-Biotin), as this end was not required for RNAi. HeLa cells were co-transfected with biotinylated EGFP duplex siRNA (SS/AS3′-Biotin) and pEGFP-C1. siRNAs were isolated by pull-out assay, and then were subjected to phosphatase and kinase reactions (J. R. Lakowicz, Principles of Fluorescence Spectroscopy (Kluwer Acadmic/Plenum Publishers, N.Y., 1999)). The mobility of siRNAs was established on a 20% polyacrylamide-7 M urea gel (FIG. 5C). FIG. 5C, lanes 1-3 (marker lanes) contain 5′ end-labeled RNA sense strands (ss) (lane 1), 3′ biotinylated antisense strands (AS3′-Biotin) (lane 2), and heat denatured (10 min at 95° C.) siRNA duplexes (SS/AS3′-Biotin) (lane 3). Isolated biotinylated siRNA are shown with (lanes 5 and 7) or without SAP treatment (lanes 6 and 8). RNA isolated as above from HeLa cells without siRNA transfection is shown in lane 4. The sense strand migrated further than AS-3′-Biotin, and both strands appeared as two distinct bands in the same lane if isolated in a duplex form (FIG. 5C; lanes 1-3).

To determine if the 5′ end of the antisense strand was being phosphorylated, streptavidin magnetic beads were used to pull out biotinylated siRNAs from cells 6 h post-transfection, were washed to remove unbound RNA, and then split into two aliquots. One aliquot was dephosphorylated with shrimp alkaline phosphatase (SAP) and the siRNA 5′ ends were labeled with ³²p by T4 polynucleotide kinase (PNK) reactions. The other aliquot was subjected to 5′ end radiolabeling by PNK without prior dephosphorylation by SAP. Cells not transfected with siRNA did not show any bands (FIG. 5C; lane 4), indicating that siRNAs recovered were specific to those transfected into cells and pulled out with streptavidin magnetic beads. In the absence of ATPA-18, efficient 5′ end radiolabeling was seen only when siRNA was pre-treated with SAP. This indicated that the 5′ ends had been phosphorylated in vivo (FIG. 5C; lanes 5-6), thereby preventing the addition of a phosphate group without prior removal of the phosphate group added in vivo. It was also apparent in the absence of the ATP analog that predominantly only the biotinylated antisense strands were pulled out from cells. These results indicated that siRNAs pulled out were mostly single stranded, signifying that the duplex had been unwound prior to removal from cells.

In the presence of 50 μM ATPA-18, which was added at the time of transfection, efficient 5′ end radiolabeling was observed only when siRNA was pre-treated with SAP (FIG. 5C; lanes 7-8), indicating that the 5′ ends had been phosphorylated in vivo, as was seen in the absence of ATPA-18. This suggested that ATPA-18 was not blocking the siRNA 5′ phosphorylation step of the RNAi pathway. However, in contrast to what was seen in the absence of ATPA-18, a significant amount of sense strand siRNA was pulled out with the biotinylated antisense strand upon treatment with ATPA-18 (FIG. 5C; lanes 7-8 arrows). The presence of both strands signified that the siRNA duplex had not been unwound at the time AS-3′-Biotin was pulled out from the cell. This result demonstrated that the ATPA-18 analog was specifically affecting the unwinding step of the RNAi pathway, suggesting that the target of ATPA-18 inhibition was an ATP-dependent RNA helicase.

### EXAMPLE 8

### Affects of RNAi Inhibitor on Unwinding in Human Cells

To directly study the effects of ATPA-18 on siRNA unwinding in vivo, a fluorescence resonance energy transfer (FRET) experiment was designed to distinguish wound and unwound siRNA. FRET, in which a fluorescent donor molecule transfers energy via a nonradiative dipole-dipole interaction to an acceptor molecule, is a spectroscopic technique for visualizing the phenomenon associated with the distance change between donors and acceptors. The efficiency of FRET (E_{FRET}) can be expressed as Equation 1:

$${E}_{FERT} = {( 1 + \frac{R}{{R}_{0}} )}^{- 6} \qquad ( 1 )$$

where R is the distance between donor and acceptor; R₀ is the Forester distance. Practically, E_{FRET} can be calculated by measuring the donor fluorescence intensity in the absence of an acceptor (ID) and in the presence of an acceptor (I_{DA}) (Equation 2).

$${E}_{FERT} = 1 - \frac{{I}_{DA}}{{I}_{D}} \qquad ( 2 )$$

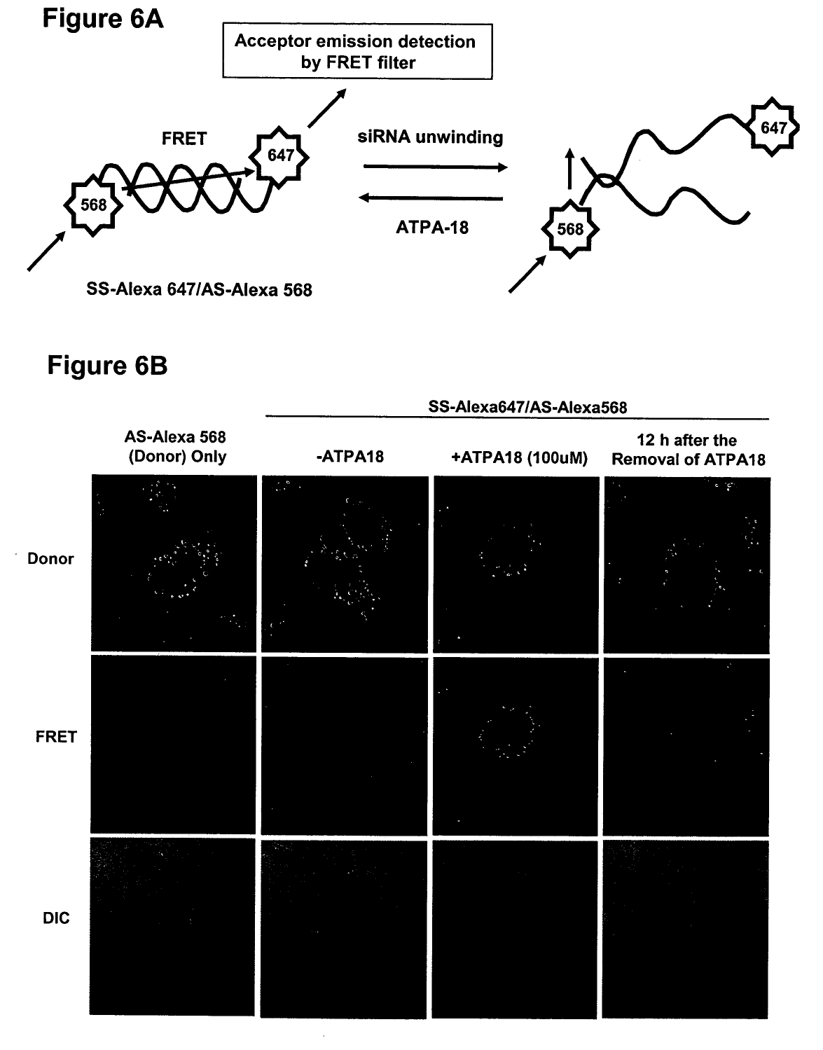

In this Example, Alexa Fluor 568 (donor) was incorporated at the antisense siRNA (AS-Alexa568) and Alexa Fluor 647 (acceptor) was incorporated at the sense siRNA (SS-Alexa647). Both donor and acceptor were conjugated at the 3′-end of each strand (FIG. 6A). FRET would only be observed when the labeled siRNA was in a duplex structure, and would not be observed when siRNA was unwound (FIG. 6A), providing an unambiguous method of assaying RNA helicase activity.

siRNA were labeled with Alexa 647 and Alexa 568 at the 3′ terminus of the siRNA sense and antisense strand, respectively. Duplex siRNA (SS-Alexa 647/AS-Alexa 568) were transfected into HeLa cells with or without ATPA-18 treatment. Transfection mixtures were removed at 6 h post transfection and cells were continuously cultured in the presence or absence of ATPA-18. Cells were imaged at 8 h post transfection. Cells were imaged with a Leica confocal imaging spectrophotometer system (TCS-SP2) attached to a Leica DMIRE inverted fluorescence microscope and equipped with an acousto-optic tunable filter (AOTF) and a tunable acousto-optical beam splitter (AOBS). A 63 X, 1.32 NA oil immersion objective was employed. For donor (AS-Alexa 568) detection, the fluorescence emission signal was recorded at 585±30 nm when excited at the Alexa 568 excitation wavelength (543 nm). For FRET signal detection, the fluorescence emission signal was recorded at 675±25 nm when excited at the Alexa 568 excitation wavelength (543 nm).

Fluorescence was observed only with the donor filter and not the FRET filter when only AS-Alexa 568 was transfected into cells (FIG. 6B; left panel). In addition, FRET was not observed when AS-Alexa 568/SS-Alexa 647 siRNA was transfected into HeLa cells in the absence of ATPA-18 (FIG. 6B; 2^{nd} panel from left), indicating that siRNA unwinding had occurred. In the presence of ATPA-18, fluorescence was observed with both the donor and FRET filters. This indicates that unwinding had not occurred and that ATPA-18 specifically inhibited siRNA unwinding in vivo, corroborating the above in vitro analysis that showed siRNA in ATPA-18 treated cells did not unwind at a high efficiency. The effect on unwinding was also reversible. After exerting its effect on unwinding, as manifested by FRET, ATPA-18 was removed from the cell media for 12 h and FRET was lost, signifying that the cell had regained the capacity to unwind siRNA.

To obtain E_{FRET} in vivo between AS-Alexa 568 and SS-Alexa 647, a second method for detecting FRET was utilized in which donor emissions lost by energy transfer during FRET were restored by deliberately photobleaching the acceptor fluorochrome, abolishing its capacity as an energy acceptor. A. K. Kenworthy, M. Edidin, J Cell Biol 142, 69-84 (Jul. 13, 1998). This observable phenomenon can be achieved by using a confocal microscope. Prior to photobleaching, the Alexa568 intensity in a cell stimulated by uniquely exciting Alexa568 (I_{pre-photobleach}) is partially quenched by Alexa647 when FRET occurs. Shining a high-intensity, acceptor-sensitive laser into an area of interest, Alexa647 fluorescence in this area will be permanently abolished and FRET will no longer be possible. The Alexa568 intensity after photobleaching (I_{post-photobleach}) will then increase significantly. For this analysis, Equation 2 is rewritten as Equation 3.

$${E}_{FERT} = 1 - \frac{{I}_{pre \text{-} photobleach}}{{I}_{post \text{-} photoblease}} \qquad ( 3 )$$

Duplex siRNAs (SS-Alexa 647/AS-Alexa 568) were prepared, transfected and analyzed for FRET with or without ATPA-18 treatment. To quantify FRET efficiency, photobleaching studies were performed with conventional excitation at 633 nm for Alexa 647. FRET efficiency was determined by measuring the ratio of the Alexa 568 (Donor) fluorescence emission signal pre- and post-photobleaching of Alexa 647 (acceptor) (panel 3 versus panel 1). Image analysis was performed using Leica Confocal Software (LCS).

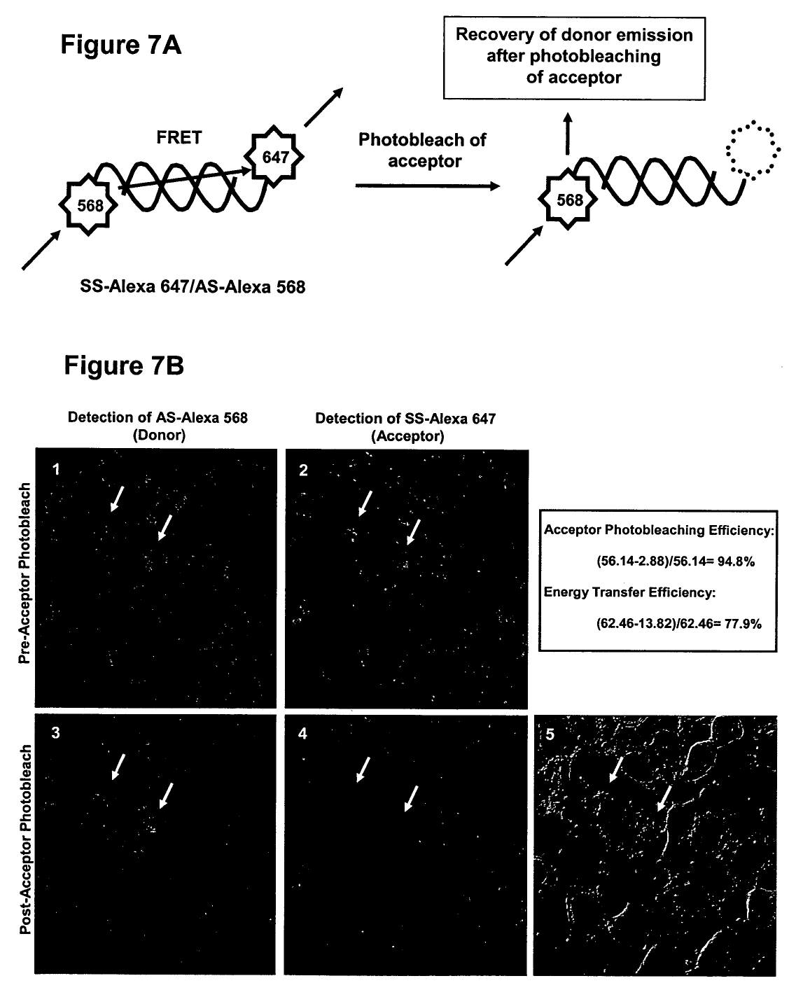

Unwound siRNA gives a very small E_{FRET} and photobleaching of the Alexa647 would not affect the fluorescence of Alexa568 (FIG. 7B). If ATPA-18 inhibits siRNA unwinding, and FRET between Alexa 568 and Alexa 647 transpires, photobleaching would result in an increment of Alexa568 fluorescence intensity (FIG. 7A) and a high E_{FRET} would be obtained.

In the absence of ATPA-18, acceptor emissions were not detected and a change in donor fluorescence was not detected post-acceptor photobleaching, indicating that FRET had not occurred and unwinding had taken place. In the presence of ATPA-18, prior to acceptor photobleaching, the donor showed dim fluorescence while the acceptor displayed strong fluorescence (FIG. 7B, left panels). Post-photobleaching, donor fluorescence became quite strong and acceptor fluorescence was no longer observed, exhibiting 94.8% photobleaching efficiency (FIG. 7B, middle panels). The energy transfer efficiency was calculated to be 77.9%, indicating that FRET occurred between AS-Alexa 568 and SS-Alexa 647, and strongly suggested that unwinding had not occurred in the presence of ATPA-18.

ATPA-18 effects on unwinding suggested that the effect of the small molecule on RNAi was specific to double-stranded siRNA and may not affect RNAi caused by single-stranded siRNA. Single-stranded siRNA is generally not as efficient at causing RNAi effects, requiring a higher siRNA concentration to cause RNAi, but still can enter the RNAi pathway (D. S. Schwarz et al., Mol Cell 10, 537-48. (2002); J. Martinez et al. Cell 110, 563-574. (2002)).

To determine if ATPA-18 effects were specific to double-stranded siRNA, an in vitro RNAi cleavage assay was performed with either double-stranded or single-stranded siRNA in the absence or presence of various concentrations of ATPA-18 (FIG. 8). The effect of ATPA-18 was specific to double-stranded siRNA and did not have an effect on RNAi caused by single-stranded siRNA. This result demonstrated that the RNA helicase activity inhibited by ATPA-18 was required for unwinding double-stranded siRNA to induce RNAi but was not required for RNAi mediated by single-stranded siRNA.

### SUMMARY OF EXAMPLES 7-8

In the above examples, two ATP analogs (ATPA-18 and ATPA-21) were identified that inhibit RNA helicase activity. Both analogs abolished RNAi in vivo. In addition, biochemical and FRET analyses demonstrated that one of the analogs inhibited siRNA unwinding. These results provided direct evidence for RNA helicase activity in the RNAi pathway and this activity was required for RNAi. The ATP analogs identified represent new, targeted probes that modulate the RNAi pathway at a discrete step, establishing a new gene knockdown approach for deciphering in vivo genetic functions through reverse genetics.

Analogs ATPA-18 and ATPA-21 were identified as powerful tools for studying RNAi since they were not toxic, had readily assayed effects on RNAi, and removal from media reversed their negative effects. This reversibility presents a new method for modulating reverse genetics using small molecules and RNAi to harness the control of gene expression and evaluate the effects of gene knockdown.

ATPA-18 was further characterized as a potent, targeted inhibitor of a discrete step of the RNAi pathway in human cells, providing the first direct evidence of in vivo ATP-dependent RNA helicase activity in human cells during RNAi. This analysis of ATPA-18 showed that RNA helicase activity could specifically be inhibited by a small molecule, and demonstrated in vivo that siRNA unwinding was ATP-dependent and essential for RNAi induction. The timing of unwinding was also established, occurring within the first 6 h of transfection. However, once unwinding occurred within 6 h of siRNA transfection, RNA helicase activity was no longer required for RNAi, indicating that the unwinding step was not reversible once completed. Both ATP analogs were unable to inhibit RNAi if cells were treated 6 h after transfection (FIG. 5A). This indicated that the step(s) of the RNAi pathway affected by ATPA-18 and ATPA-21 occurred between 0-6 h post-transfection, and once completed within this window of time was an irreversible part of the pathway.

Another important outcome of these examples was the demonstration of a direct correlation between RNA helicase activity, completion of siRNA unwinding, and downstream RNAi events like mRNA cleavage. A significant implication of this connection is that unwinding of siRNA may be an essential prerequisite for mRNA cleavage. This could explain why double-stranded siRNA was more effective at inducing RNAi than single-stranded siRNA (FIG. 4B). Not participating in unwinding may preclude single-stranded siRNA from being efficiently incorporated into the RNAi pathway. That is, recognition and interactions between double-stranded siRNA and the RNA helicase may trigger the activation of downstream RNAi events or direct interactions with downstream components of the RNAi pathway.

Several key findings highlighted herein arose from using small molecules as probes to study intracellular mechanisms, representing a new approach for targeting and thereby delineating discrete steps in a particular pathway like RNAi. The chemical library was synthesized to target ATP-dependent steps within a particular pathway, which in this case was the RNAi pathway. Having a method, like the dual fluorescence assay, for measuring RNAi activity and knowing the steps likely targeted by the small molecules dictated the analysis of the probes once they were isolated in the screen. For these reasons, this new approach of synthesizing and screening small molecules is exceptionally directed, and increases the pace of identifying mechanistic targets of the small molecule.

### EXAMPLE 9

### Affinity Purification of RNAi Components

Candidate compounds can be used to affinity purify components involved in RNAi pathways in various organisms. The purified proteins can provide insight into the mechanism of RNAi in mammalian cells, and results could guide the design of siRNA structures useful in probing biological questions and in functional genomic studies. An affinity purification can be carried out as follows.

Immobilization on Magnetic Bead

Biotin conjugated small ligand prepared with at least one compound of the invention are bound to 50 μl of streptavidin-coated magnetic beads (Dynal Inc.) by incubating 300 pmole ligand and 50 μl beads in 200 μl TK buffer (20 mM KCl, 50 mM Tris-HCl, pH 7.4, 0.1% TRITON X-100, 0.1% BSA) at room temperature for 4 h on a shaker. Ligand-bound beads are washed 4 times with 600 μl TK buffer and then equilibrated with reporter lysis buffer (Promega) and stored at 4° C. until use.

Purification of IP and SKP Analog Binding Proteins from Crude Cell Extract

HeLa cells grown on 100 mm plate with 90% confluency are lysed in ice-cold reporter lysis buffer (Promega) containing protease inhibitor (complete, EDTA-free, 1 tablet/10 mL buffer, Roche Molecular Biochemicals). The resulting lysates are cleared by centrifugation and protein amount in the clear lysate are quantified by Dc protein assay kit (Bio-Rad). 600 μg of total cell lysate in 200 μl repoter lysis buffer are incubated with 50 μl ligand bound beads and rocked at 4° C. for 12 h. The cell lysate treated beads are then washed 4 times with 600 μl reporter lysis buffer containing 0.1% TRITON X-100 and 0.1% BSA. Proteins that bind to the ligand are released from the magnetic beads by boiling in 30 ul 1×SDS loading buffer (50 mM Tris-HCl, pH 6.8, 12% glycerol, 4% SDS, 100 mM DTT, and 0.01% Coomassie Blue G-250) and resolved in 10% SDS-PAGE followed by silver stain (Bio-rad). Further characterization of the identified proteins is carried out using methods well known in the art, e.g., peptide mapping, microsequencing.

Equivalents

Those skilled in the art will recognize, or be able to ascertain using no more than routine experimentation, numerous equivalents to the specific procedures described herein. Such equivalents are considered to be within the scope of the present invention and are covered by the following claims. The contents of all references, issued patents, and published patent applications cited throughout this application are hereby incorporated by reference. The appropriate components, processes, and methods of those patents, applications and other documents may be selected for the present invention and embodiments thereof.

We claim:

1. A compound for modulating RNAi having the formula (I): 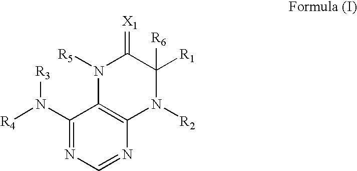
wherein
R¹ is alkyl, alkenyl, or alkynyl, optionally interrupted by one or more O, N, NR^{a}, or S groups, and optionally substituted with one or more hydroxyl, halo, alkoxy, oxo, amino, cycloalkyl, heterocycloalkyl, aryl, heteroaryl, thione or thiol moiety, the cycloalkyl, heterocycloalkyl, aryl, or heteroaryl is optionally substituted with one or more oxo, hydroxyl, thione, thiol or fused ring moiety;
R² is H or C₁₋₆ alkyl, or R¹ and R² together form a 3- to 8-membered ring optionally interrupted by one or more O, NR^{a}, or S and optionally substituted with one or more hydroxyl, halo, alkoxy, oxo, amino, thione or thiol moiety;
R₃ is alkyl, alkenyl, alkynyl, aryl, cycloalkyl, or heterocycloalkyl, optionally interrupted by one or more O, NR^{a}, or S group, and optionally substituted with one or more hydroxyl, alkoxy, halo, aryl, heteroaryl, cycloalkyl, heterocycloalkyl, carboxyl, alkylcarboxy, oxo, amino, aminoaryl, aminoheteroaryl, indole, alkoxyaryl, alkoxycarbonyl, thione, thiol, or a fused ring moiety; and
R₄ is H or C₁₋₆ alkyl optionally substituted with a hydroxyl, halo, or amino group, or R³ and R⁴ together form a 3- to 8-membered ring optionally interrupted by one or more O, NR^{a}, or S and optionally substituted with one or more alkyl, hydroxyl, alkoxy, halo, aryl, heteroaryl, cycloalkyl, heterocycloalkyl, carboxyl, alkylcarboxy, oxo, amino, indole, thione, thiol or a fused ring moiety;
R⁵is H or C₁₋₆ alkyl;
R⁶ is H or C₁₋₆ alkyl;
X¹ is O, S or NR^{a}; and
R^{a} is H, C₁₋₆ alkyl, or C₁₋₆ acyl,
or a salt thereof.

2. A compound for modulating RNAi having the formula (II): 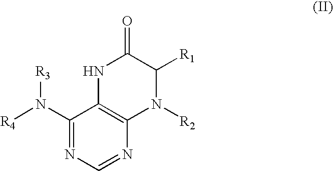
wherein
R¹ is alkyl, alkenyl, or alkynyl, optionally interrupted by one or more O, N, NR^{a}, or S groups, and optionally substituted with one or more hydroxyl, halo, alkoxy, oxo, amino, cycloalkyl, heterocycloalkyl, aryl, heteroaryl, thione or thiol moiety, the cycloalkyl, heterocycloalkyl, aryl, or heteroaryl optionally substituted with one or more oxo, hydroxyl, thione, thiol or fused ring moiety;
R² is H or C₁₋₆ alkyl, or R¹ and R² together form a 3- to 8-membered ring optionally interrupted by one or more O, NR^{a}, or S and optionally substituted with one or more hydroxyl, halo, alkoxy, oxo, amino, thione or thiol moiety;
R₃ is alkyl, alkenyl, alkynyl, aryl, cycloalkyl, or heterocycloalkyl, optionally interrupted by one or more O, NR^{a}, or S group, and optionally substituted with one or more hydroxyl, alkoxy, halo, aryl, heteroaryl, cycloalkyl, heterocycloalkyl, carboxyl, alkylcarboxy, oxo, amino, aminoaryl, aminoheteroaryl, indole, alkoxyaryl, alkoxycarbonyl, thione, thiol, or a fused ring moiety; and
R₄ is H or C₁₋₆ alkyl optionally substituted with a hydroxyl, halo, or amino group, or R³ and R⁴ together form a 3- to 8-membered ring optionally interrupted by one or more O, NR^{a}, or S and optionally substituted with one or more alkyl, hydroxyl, alkoxy, halo, aryl, heteroaryl, cycloalkyl, heterocycloalkyl, carboxyl, alkylcarboxy, oxo, amino, indole, thione, thiol or a fused ring moiety;
R^{a} is H, C₁₋₆ alkyl, or C₁₋₆ acyl,
or a salt thereof.

3. The compound of claim 2, wherein
R¹ is alkyl, wherein R¹ is optionally interrupted by one or more O, N, or NH groups, and R¹ is optionally substituted with one or more hydroxyl, oxo, amino, cycloalkyl, heterocycloalkyl, aryl, or heteroaryl moiety, the cycloalkyl, heterocycloalkyl, aryl, or heteroaryl optionally substituted with one or more oxo, thione or fused ring moiety;
R² is H, or R¹ and R² together form a 5- to 7-membered ring optionally substituted with one or more hydroxyl or amino moiety;
R₃ is alkyl, cycloalkyl, or heterocycloalkyl, optionally interrupted by one or more NR^{a} group, and optionally substituted with one or more hydroxyl, aryl, heteroaryl, cycloalkyl, heterocycloalkyl, amino, aminoaryl, aminoheteroaryl, indole, alkoxyaryl, or a fused ring moiety;
R₄ is H or C₁₋₆ alkyl optionally substituted with a hydroxyl group, or R³ and R⁴ together form a 5- to 7-membered ring optionally interrupted by one or more O or NH, and optionally substituted with one or more alkyl, hydroxyl, alkoxy, carboxyl, alkylcarboxy, oxo, indole, or a fused ring moiety; and
R^{a} is H or C₁₋₆ alkyl;
or a salt thereof.

4. The compound of claim 2, wherein the compound is an RNAi activator.

5. The compound of claim 2, wherein R₁ and R₂ together form a 3-hydropyrrolidine.

6. The compound of claim 5, wherein the compound is selected from the group consisting of:

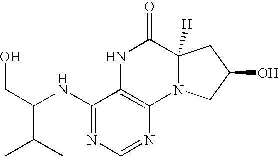

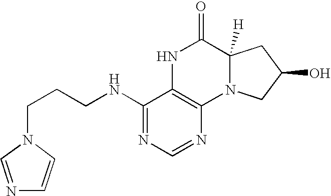

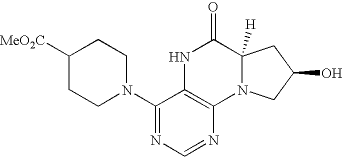

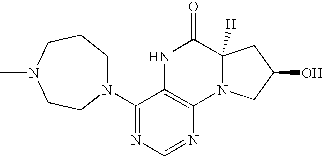

7. The compound of claim 2, wherein the compound is an RNAi inhibitor.

8. The compound of claim 7, wherein the compound is selected from the group consisting of:

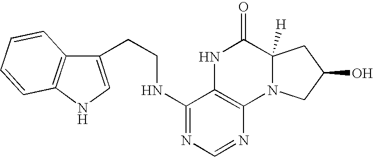

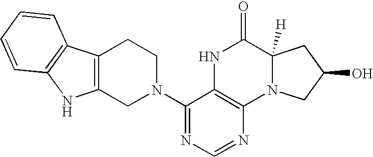

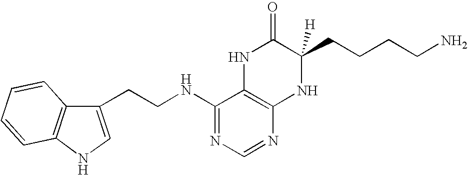

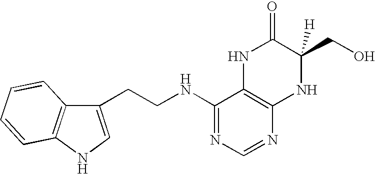

9. The compound of claim 4, wherein the compound is a trifunctionalized compound.

10. The compound of claim 8, wherein the compound is a trifunctionalized compound selected from the group consisting of:

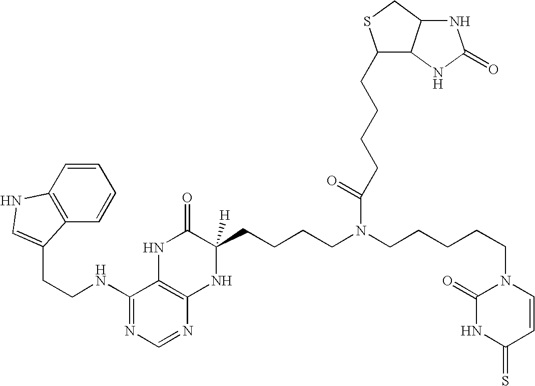

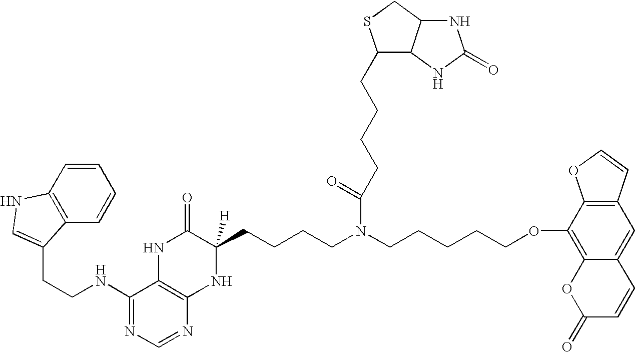

11. A pharmaceutical composition comprising the compound of any of claims 1-10 and a pharmaceutically acceptable carrier.

12. The composition of claim 11, further comprising at least one RNAi agent comprising a sequence having sufficient complementarity to at least one target RNA sequence.

13. The composition of claim 11, further comprising at least one expression vector encoding at least one RNAi agent comprising a sequence having sufficient complementarity to at least one target RNA sequence.

14. The composition of claim 13, wherein the RNAi agent is a siRNA.

15. The composition of claim 13, wherein the RNAi agent is a single stranded siRNA.

16. The composition of claim 13, wherein the RNAi agent is a double stranded siRNA.

17. The composition of claim 13, wherein the RNAi agent is modified to enhance stability.

18. The composition of claim 13, wherein the RNAi agent is modified to enhance cellular uptake.

19. A method of identifying an RNAi modulator, comprising the steps of:
contacting a cell or cell extract comprising a target gene which encodes a target protein with a RNAi agent targeted against the target gene and a candidate compound; and
detecting a measure of the target protein in the cell or cell extract, wherein a variation in the measure of the target protein against a suitable control identifies the candidate compound as an RNAi modulator.

20. The method of claim 19, wherein the candidate compound is an ATP analog.

21. The method of claim 19, wherein the target gene is a cellular gene.

22. The method of claim 19, wherein the target gene is a reporter gene.

23. The method of claim 22, wherein the reporter gene encodes a fluorescent reporter protein or polypeptide.

24. The method of claim 19, wherein the cell or cell extract further comprises a reference gene encoding a reference protein or polypeptide.

25. The method of claim 23, wherein the cell or cell extract further comprises a reference gene encoding a fluorescent reference protein or polypeptide.

26. The method of claim 24, wherein the measure of the reporter protein is a ratio of a level or activity of the reporter protein to a level or activity of a reference protein.

27. The method of claim 24, wherein the suitable control is a cell comprising the reporter gene contacted with an antisense molecule targeted against the reporter gene or an siRNA targeted against the reporter gene.

28. A method of modulating RNAi in a cell comprising the step of contacting the cell with an RNAi modulating compound or composition of any of claims 1-18 such that RNAi is modulated in the cell.

29. A method of modulating RNAi in an organism comprising the step of contacting the cell or organism with an RNAi modulating compound or composition of any of claims 1-18 such that RNAi is modulated in the cell

30. A method of modulating RNAi in a subject comprising the step of administering to the subject an RNAi modulating compound or composition of any of claims 1-18 such that RNAi is modulated in the subject.

31. A method of treating a subject at risk for or having a disease or disorder associated with normal or aberrant expression of a gene, the method comprising administering to the subject an RNAi modulating compound or composition of any of claims 1-18, such that RNAi against the gene is modulated.

32. The method of claim 31, wherein said disease or disorder is selected from a group consisting of cellular growth or proliferative disorders, skin disorders, viral infections, and gene mutation disorders.

33. The method of claim 32, further comprising the step of administering a second treatment.

34. The method of claim 33, wherein said second treatment is selected from the group consisting of radiation, chemotherapy, transfusion, and drug therapy.

35. A method for analyzing a gene expression profile in a cell or organism comprising the steps of:
contacting the cell or organism with an RNAi modulating compound or composition of any of claims 1-18; and
analyzing the affect of the RNAi modulating compound or composition on a gene expression profile in the cell or organism.

36. A method of analyzing the RNAi modulation pathway in a cell or organism comprising the steps of:
contacting the cell or the organism with an RNAi modulating compound or composition of any of claims 1-18; and
analyzing the affect of the RNAi modulating compound or composition on the RNAi modulation pathway.

37. The method of claim 36, wherein the RNAi modulating compound is ligated to an extraction media.

38. The method of claim 36, wherein the RNAi modulating compound is trifunctionalized.

39. The method of claim 36, comprising the step of contacting the cell or organism with the composition or compound at a plurality of times and locations in the cell or organism.

40. A cell or organism exhibiting a target-gene specific knock-out or knock-down phenotype comprising an RNAi modulating compound or composition of any of claims 1-18.

41. The knock-down or knock-out cell or organism of claim 40, further comprising an exogenous RNAi agent comprising a sequence having sufficient complementarity to a target gene to mediate target-specific RNAi.

42. The knock-down or knock-out cell or organism of claim 40, further comprising an expression vector encoding an exogenous RNAi agent capable of inhibiting expression of at least one target gene.

43. A method of making a compound by liquid synthesis comprising the steps of:
contacting a 4,6-dihalo-5-nitro-pyrimidine with an alpha-amino ester to form a 4-amino-6-halo-5-nitro-pyrimidine;
contacting the a 4-amino-6-halo-5-nitro-pyrimidine with a primary amine or a secondary amine to form a 4-amino-6-amino-5-nitro-pyrimidine; and
reducing the 4-amino-6-amino-5-nitro-pyrimidine to form a dihydropteridinone.

44. The method of claim 43, wherein the dihydropteridinone is a compound having the formula (I): 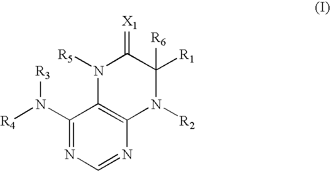
wherein
R¹ is alkyl, alkenyl, or alkynyl, optionally interrupted by one or more O, NR^{a}, or S groups, and optionally substituted with one or more hydroxyl, halo, alkoxy, oxo, amino, cycloalkyl, heterocycloalkyl, aryl, heteroaryl, thione or thiol moiety, the cycloalkyl, heterocycloalkyl, aryl, or heteroaryl is optionally substituted with one or more oxo, hydroxyl, thione, thiol or fused ring moiety;
R² is H or C₁₋₆ alkyl, or R¹ and R² together form a 3- to 8-membered ring optionally interrupted by one or more O, NR^{a}, or S and optionally substituted with one or more hydroxyl, halo, alkoxy, oxo, amino, thione or thiol moiety;
R₃ is alkyl, alkenyl, alkynyl, aryl, cycloalkyl, or heterocycloalkyl, optionally interrupted by one or more O, NR^{a}, or S group, and optionally substituted with one or more hydroxyl, alkoxy, halo, aryl, heteroaryl, cycloalkyl, heterocycloalkyl, carboxyl, alkylcarboxy, oxo, amino, aminoaryl, aminoheteroaryl, indole, alkoxyaryl, alkoxycarbonyl, thione, thiol, or a fused ring moiety; and
R₄ is H or C₁₋₆ alkyl optionally substituted with a hydroxyl, halo, or amino group, or R³ and R⁴ together form a 3- to 8-membered ring optionally interrupted by one or more O, NR^{a}, or S and optionally substituted with one or more alkyl, hydroxyl, alkoxy, halo, aryl, heteroaryl, cycloalkyl, heterocycloalkyl, carboxyl, alkylcarboxy, oxo, amino, indole, thione, thiol or a fused ring moiety;
R⁵ is H or C₁₋₆ alkyl;
R¹ is H or C₁₋₆ alkyl;
X¹ is O, S or NR^{a}; and
R^{a} is H, C₁₋₆ alkyl, or C₁₋₆ acyl,
or a salt thereof.
